(19) United States
(12) Patent Application Publication
(10) Pub. No.: US 2022/0249534 A1
(43) Pub. Date: Aug. 11, 2022

(54) METHOD OF TREATING CANCER WITH NUCLEOTIDE THERAPEUTICS

(71) Applicants: Massachusetts Institute of Technology, Cambridge, MA (US); The General Hospital Corporation, Boston, MA (US)

(72) Inventors: Matthew G. Vander Heiden, Belmont, MA (US); Peggy P. Hsu, Ann Arbor, MI (US); Frances Flewelling Diehl, Baltimore, MD (US); David B. Sykes, Cambridge, MA (US)

(21) Appl. No.: 17/650,298

(22) Filed: Feb. 8, 2022

Related U.S. Application Data
(60) Provisional application No. 63/148,490, filed on Feb. 11, 2021.

(51) Int. Cl. A61K 31/7076; A61K 31/505; A61P 35/00

(52) U.S. Cl. CPC A61K 31/7076; A61P 35/00; A61K 31/505

(57) ABSTRACT

The present disclosure provides methods for inhibiting cell proliferation, inducing differentiation, and inducing replication stress in a cancer cell. The present disclosure also provides methods for treating a cancer in a patient. Various methods of the disclosure include contacting a cancer cell with, or administering to a patient having cancer, an agent that can change the internal baseline ratio of purine:pyrimidine in the cancer cell.

### CROSS REFERENCE TO RELATED APPLICATIONS

This application claims the priority benefit of U.S. Provisional Patent Application Ser. No. 63/148,490, filed Feb. 11, 2021. The entirety of this application is hereby incorporated by reference for all purposes.

### STATEMENT REGARDING FEDERALLY SPONSORED RESEARCH OR DEVELOPMENT

This invention was made with Government support under Grant No. R01 CA201276 awarded by the National Institutes of Health (NIH). The Government has certain rights in the invention.

### FIELD

The present disclosure is in the field of medicine. More particularly, the disclosure relates to cancer treatment by creating an imbalance of purines and/or pyrimidines in the cancer cell.

### BACKGROUND

Cancer and other proliferative disorders affect many people throughout the world. True treatments for these disorders remain elusive.

Current therapies for cancer include, for example, surgical resection, radiation, chemotherapy, immunotherapy, targeted therapy and hormone therapy.

Use of surgical therapies is limited in that it is dependent on the type of cancer, its location in the body, as well as the stage of cancer. Radiation therapy is commonly used in approximately 50% of cancer patients to slow cancer growth and/or shrink the size of a tumor. It is often used in conjunction with surgery and chemotherapy. Surgery and radiation are limited in that they only treat locality or local disease. Also, radiation dose must be limited to prevent damage to normal surrounding tissue.

Antiproliferative agents such as chemotherapeutic drugs have also been used for cancer treatment. Chemotherapy works by interfering with different phases of the cell cycle or intercalating with the DNA of the cancer cell. Chemotherapy affects all tissues of the body since it is given systemically, and different chemotherapeutic agents affect different tissues and organs differently. As with other therapeutic modalities, it can be used with curative intent, along with radiation, or as a palliative measure. However, chemotherapeutic compounds non-selectively affect any actively proliferating cells, by interfering in cellular metabolism. This can result in a range of toxicities including bone marrow suppression, GI-toxicity (by affecting intestine epithelial cells or crypt cells), and hair loss (by affecting hair follicles). In addition, rapidly dividing lymphocytes, which are critical in controlling infection and in cancer surveillance, may also be suppressed, placing recipient patients at elevated risk for opportunistic infections as well as neoplasia.

Immunotherapies developed include monoclonal antibody therapies, adoptive cell transfer, cytokine therapies, vaccines and Baciilus Calmette-Guerin (BCG) therapy. Antibody therapies target molecules typically expressed on the surface of the cancer cells (such as CTLA4 and CD20). Unfortunately, some antibody based therapeutics have significant drawbacks. For example, the anti-CD20 antibody, Rituximab (Rittman), which is widely used in the treatment of B-cell malignancies leads to cell death via antibody-dependent cellular cytotoxicity (ADCC) when it binds to CD20 expressed on a B-cell surface. However, Rituximab is also known to cause significant side effects, such as headache and back pain. In addition, it possesses a slow infusion rate (50 mg/hr), and therefore administration can take up to eight hours. In addition, Rituximab increases the risk of infections and malignancies as it depletes normal B-cells as well. In fact, normal B-cell functions are essentially absent in patients that are being treated with Rituximab. Treatments like Intravenous Immune Globulin (IVIG) are well known to partially restore B-cell functions, but IVIG is a method that also has severe toxicity liabilities.

Other cancer therapeutics include small molecule drugs that can be amenable to oral administration. These, small molecule cancer drugs target cell surface ligand-binding receptors as well as the intracellular proteins, including metabolic enzymes and anti-apoptotic proteins that play a key role in transducing downstream signaling for cell growth and metastasis promotion. However, the activity of these small molecule therapies also can affect any such pathways in non-cancerous cells they encounter.

Thus, as standard cancer therapies each have significant limitations, and none are curative, improved cancer therapeutics are still needed.

### SUMMARY

It has been discovered that the addition of certain (individual types of) purines and/or pyrimidines to a cancer cell, thus causing a nucleotide imbalance in the cell, leads to replication stress, inhibition of proliferation, and the induction of cell differentiation.

These discoveries have been exploited to develop the present disclosure, which, in one aspect, is directed to a method of inhibiting proliferation in a cancer cell, the cancer cell having an endogenous baseline ratio of purine:pyrimidine, the method comprising: contacting the cell with an agent in an amount sufficient to change the endogenous baseline ratio of purine:pyrimidine in the cell, resulting in a nucleotide imbalance, the agent comprising a purine, a purine precursor, a purine analog that is not a purine biosynthesis inhibitor, a pyrimidine, a pyrimidine precursor, and/or a pyrimidine analog that is not a pyrimidine biosynthesis inhibitor, the nucleotide imbalance inhibiting the cancer cell from proliferating.

In an exemplary embodiment, the agent is a purine nucleotide. In some embodiments, the purine nucleotide is Adenine or Guanine. In an exemplary embodiment, the agent is Adenine.

In another embodiment, the agent is a purine precursor. In certain embodiments, the purine precursor is AIR, CAIR, SACAIR, AICAR, FAICAR inosine mono phosphate (IMP), adenylosuccinate, xanthine, or hypoxanthine. In particular embodiments, the agent is a purine analog that does not inhibit purine biosynthesis. In a specific embodiment, the purine analog is 8-amino-adenosine.

In an exemplary embodiment, the cancer cell is contacted with at least one purine and at least one purine precursor. In other embodiments, the cancer cell is further contacted with at least on purine analog that is not an inhibitor of purine biosynthesis. In some embodiments, the method further comprises contacting the cancer cell with a pyrimidine biosynthesis inhibitor. In particular embodiments, the pyrimidine biosynthesis inhibitor is mercaptopurine, 6-mercaptopurine, mycophenolic acid, mycophenolate mofetil, 6-thioguanine, lometrexol, pyrimethamine, or cladribine. In particular embodiments,

In other exemplary embodiments, the agent is a pyrimidine nucleotide. In some embodiments, the pyrimidine nucleotide is Cytosine, Thymidine, or Uracil. In an exemplary embodiment, the agent is a pyrimidine precursor. In certain embodiments, the pyrimidine precursor is dihydroorotate, orotate, uracil monophosphate (UMP), UDP, CMP, or CDP. In other embodiments, the agent is a pyrimidine analog that does not inhibit pyrimidine biosynthesis. In specific embodiments, the pyrimidine analog is cytarabine, nalarabine, sapacitabine, ARC (4-amino-6-hydrazino-7-β-D-ribofuranosyl-7H-pyrrolo[2,3-d]-pyrimidine-5-carboxamide). In some embodiments, the cancer cell is contacted with at least one pyrimidine and at least one pyrimidine precursor. In an exemplary embodiment, the cancer cell is further contacted with at least on pyrimidine biosynthesis inhibitor that is not a pyrimidine biosynthesis inhibitor. In another embodiment, the method further comprises contacting the cancer cell with a purine biosynthesis inhibitor. In certain embodiments, the purine biosynthesis inhibitor is Azathioprine, Mercaptopurine, Clofarabine, Thioguanine, Fludarabine, Pentostatin, Cladribine or Acycloguanosine. In an exemplary embodiment, the method further comprises contacting the cells with a pyrimidine biosynthesis inhibitor. In certain embodiments, the pyrimidine biosynthesis inhibitor is brequinar, leflunomide, teriflunomide, pyrazofurin, cyclopentenyl cytosine, fluorocyclopentenylcytosine, 5-fluorouracil, ralitrexed, pemetrexed, or 6-azauridine.

In an exemplary embodiment, the cancer cell is a liquid cancer cell or a solid cancer cell.

In another aspect, the present disclosure is directed to a method of inducing differentiation in a cancer cell, the cancer cell having an endogenous baseline ratio of purine:pyrimidine, the method comprising: contacting the cell with an agent in an amount sufficient to change the endogenous baseline ratio of purine:pyrimidine in the cell, resulting in a nucleotide imbalance, the agent comprising a purine, a purine precursor, a purine analog that is not a purine biosynthesis inhibitor, a pyrimidine, a pyrimidine precursor, and/or a pyrimidine analog that is not a pyrimidine biosynthesis inhibitor, the nucleotide imbalance inducing differentiation in the cancer cell.

In some embodiments, the agent is a purine nucleotide. In certain embodiments, the purine nucleotide is Adenine or Guanine. In an exemplary embodiment, the agent is Adenine. In some embodiments, the agent is a purine precursor. In specific embodiment, the purine precursor is AIR, CAIR, SACAIR, AICAR, FAICAR inosine mono phosphate (IMP), adenylosuccinate, xanthine, or hypoxanthine. In some embodiment, the agent is a purine analog that is not a purine biosynthesis inhibitor. In specific embodiments, the purine analog is 8-amino-adenosine. In some embodiments, the cancer cell is contacted with at least one purine and at least one purine precursor.

In an exemplary embodiment, the cancer cell is further contacted with at least on purine analog that is not an inhibitor of purine biosynthesis. In another embodiment, the method further comprises contacting the cancer cell with a pyrimidine biosynthesis inhibitor. In certain embodiments, the pyrimidine biosynthesis inhibitor is mercaptopurine, 6-mercaptopurine, mycophenolic acid, mycophenolate mofetil, 6-thioguanine, lometrexol, pyrimethamine, or cladribine. In an exemplary embodiment, the agent is a pyrimidine nucleotide, in some embodiment, the pyrimidine nucleotide is Cytosine, Thymidine, or Uracil. In particular embodiments, the agent is a pyrimidine precursor. And in certain embodiments, the pyrimidine precursor is dihydroorotate, orotate, uracil monophosphate (UMP), UDP, CMP, or CDP. In some embodiments, the agent is a pyrimidine analog that is not an inhibitor of pyrimidine biosynthesis. In specific embodiments, the pyrimidine analog is cytarabine, nalarabine, sapacitabine, ARC (4-amino-6-hydrazino-7-β-D-ribofuranosyl-7H-pyrrolo[2,3-d]-pyrimidine-5-carboxamide). In an exemplary embodiment, the cancer cell is contacted with at least one pyrimidine and at least one pyrimidine precursor. In some embodiments, the cancer cell is further contacted with at least on pyrimidine analog that is not an inhibitor of pyrimidine biosynthesis. In some embodiments, the method further comprises contacting the cell with a purine biosynthesis inhibitor. In certain exemplary embodiments, the purine biosynthesis inhibitor is Azathioprine, Mercaptopurine, Clofarabine, Thioguanine, Fludarabine, Pentostatin, Cladribine, or Acycloguanosine. In particular embodiments, the method further comprises contacting the cancer cell with a pyrimidine biosynthesis inhibitor. In exemplary embodiments, the pyrimidine biosynthesis inhibitor is brequinar, leflunomide, teriflunomide, pyrazofurin, cyclopentenyl cytosine, fluorocyclopentenylcytosine, 5-fluorouracil, ralitrexed, pemetrexed, or 6-azauridine.

In an exemplary embodiment, the cancer cell is a liquid cancer cell or a solid cancer cell.

In another aspect, the present disclosure is directed to a method of inducing replication stress in a cancer cell, the cancer cell having an endogenous baseline ratio of purine:pyrimidine, the method comprising: contacting the cell with an agent in an amount sufficient to change the endogenous baseline ratio of purine:pyrimidine in the cell, resulting in a nucleotide imbalance, the agent comprising a purine, a purine precursor, a purine analog that is not a purine biosynthesis inhibitor, a pyrimidine, a pyrimidine precursor, and/or a pyrimidine analog that is not a pyrimidine biosynthesis inhibitor, the nucleotide imbalance inducing replication stress in the cancer cell.

In some embodiments, the agent is a purine nucleotide. In certain embodiments, the purine nucleotide is Adenine or Guanine. In an exemplary embodiment, the agent is Adenine. In other embodiments, the agent is a purine precursor. In some embodiments, the purine precursor is AIR, CAIR, SACAIR, AICAR, FAICAR inosine mono phosphate (IMP), adenylosuccinate, xanthine, and hypoxanthine. In another embodiment, the agent is a purine analog that is not a purine biosynthesis inhibitor, and in a specific exemplary embodiment, the purine analog is 8-amino-adenosine. In other embodiments, the cancer cell is contacted with at least one purine, and at least one purine precursor.

In some embodiments, the cancer cell is further contacted with at least on purine analog that is not an inhibitor of purine biosynthesis. In certain embodiments, the method further comprises contacting the cancer cell with a pyrimidine biosynthesis inhibitor. In specific embodiments, the pyrimidine biosynthesis inhibitor is mercaptopurine, 6-mercaptopurine, mycophenolic acid, mycophenolate mofetil, 6-thioguanine, lometrexol, pyrimethamine, or cladribine. In exemplary embodiments, the agent is a pyrimidine nucleotide, and in particular embodiments, the pyrimidine nucleotide is Cytosine, Thymidine, or Uracil. In other embodiments, the agent is a pyrimidine precursor, and in certain embodiments, the pyrimidine precursor is dihydroorotate, orotate, uracil monophosphate (UMP), UDP, CMP, or CDP.

In an exemplary embodiment, the agent is a pyrimidine analog that is not a pyrimidine biosynthesis inhibitor. In some exemplary embodiment, the pyrimidine analog is cytarabine, nalarabine, sapacitabine, ARC (4-amino-6-hydrazino-7-β-D-ribofuranosyl-7H-pyrrolo[2,3-d]-pyrimidine-5-carboxamide). In other embodiments, further comprises contacting the cancer cell with a purine biosynthesis inhibitor. In certain embodiment, the purine biosynthesis inhibitor is brequinar, leflunomide, teriflunomide, pyrazofurin, cyclopentenyl cytosine, fluorocyclopentenylcytosine, 5-fluorouracil, ralitrexed, pemetrexed, or 6-azauridine. In some embodiments, the cancer cell is contacted with at least one pyrimidine and at least one pyrimidine precursor. In another embodiment, the cancer cell is further contacted with at least on pyrimidine analog that is not a purine biosynthesis inhibitor. In certain embodiments, the pyrimidine analog is cytarabine, nalarabine, sapacitabine, ARC (4-amino-6-hydrazino-7-β-D-ribofuranosyl-7H-pyrrolo[2,3-d]-pyrimidine-5-carboxamide). In other embodiments, the cancer cell is further contacted with a purine synthesis inhibitor. In specific embodiment, the purine biosynthesis inhibitor is Azathioprine, Mercaptopurine, Clofarabine, Thioguanine, Fludarabine, Pentostatin, Cladribine, or Acycloguanosine

In an exemplary embodiment, the cancer cell is a liquid cancer cell or a solid cancer cell.

In another aspect, the present disclosure is directed to a method of treating a subject afflicted with a cancer, comprising: administering to the subject a therapeutically effective amount of an agent that changes the endogenous baseline purine:pyrimidine ratio in a cell of the cancer, thereby causing a nucleotide imbalance in the cancer, the agent comprising a purine, a purine precursor, a purine analog that is not a purine biosynthesis inhibitor, a pyrimidine, a pyrimidine precursor, and/or a pyrimidine analog that is not a pyrimidine biosynthesis inhibitor, the nucleotide imbalance resulting in a reduction in, and/or inhibition of proliferation of the cancer. In other embodiments, the agent is a purine nucleotide. In particular embodiments, the purine nucleotide is Adenine or Guanine, and in an exemplary embodiment, the agent is Adenine. In other embodiments, the agent is a purine precursor. In certain embodiments, the purine precursor is AIR, CAIR, SACAIR, AICAR, FAICAR inosine mono phosphate (IMP), adenylosuccinate, xanthine, or hypoxanthine. In some embodiments, the agent is a purine analog that is not a purine biosynthesis inhibitor. In an exemplary embodiment, the purine analog is 8-amino-adenosine. In other embodiment, the cancer cell is contacted with at least one purine, and at least one purine precursor.

In an exemplary embodiment, the cancer cell is further contacted with at least on purine analog that is not an inhibitor of purine biosynthesis. In some embodiments, the method further comprises contacting the cancer cell with a pyrimidine biosynthesis inhibitor. In specific embodiments, the pyrimidine biosynthesis inhibitor is mercaptopurine, 6-mercaptopurine, mycophenolic acid, mycophenolate mofetil, 6-thioguanine, lometrexol, pyrimethamine, or cladribine. In other embodiments, the agent is a pyrimidine nucleotide. In certain embodiments, the pyrimidine nucleotide is Cytosine, Thymidine, or Uracil. In another an exemplary embodiment, the agent is a pyrimidine precursor. Sin specific embodiments, the pyrimidine precursor is dihydroorotate, orotate, uracil monophosphate (UMP), UDP, CMP, or CDP.

In an exemplary embodiment, the agent is a pyrimidine analog that does not affect pyrimidine biosynthesis. In certain, the pyrimidine analog is cytarabine, nalarabine, sapacitabine, ARC (4-amino-6-hydrazino-7-β-D-ribofuranosyl-7H-pyrrolo[2,3-d]-pyrimidine-5-carboxamide). In other embodiments, the cancer cell is contacted with at least one pyrimidine and at least one pyrimidine precursor. In some embodiments, the method further comprises contacting the cell with at least one pyrimidine analog that is not a pyrimidine biosynthesis inhibitor. In another embodiment, the cancer cell is further contacted with at least one purine biosynthesis inhibitor. In certain embodiments, the purine biosynthesis inhibitor is Azathioprine, Mercaptopurine, Clofarabine, Thioguanine, Fludarabine, Pentostatin, Cladribine or Acycloguanosine.

In an exemplary embodiment, the cancer is a liquid cancer or a solid cancer.

In some embodiments, the agent is in a formulation.

### DESCRIPTION OF THE DRAWINGS

The foregoing and other objects of the present disclosure, the various features thereof, as well as the disclosure itself may be more fully understood from the following description, when read together with the accompanying drawings in which:

FIG. 1A is a graphic representation of the proliferation rate of RPE-1 cells incubated with a single concentration of the indicated nucleotide precursors;

FIG. 1B is a graphic representation of the, proliferation rate of A549 cells incubated with a single concentration of the indicated purines or nucleosides;

FIG. 2A is a graphic representation of GTP levels in A549 cells incubated with the nucleotide precursors Guanine (A) or a combination of Guanine and adenine (G+A);

FIG. 2B is a graphic representation of ATP levels in A549 cells treated with the nucleotide precursors Guanine (A) or a combination of Guanine and adenine (G+A);

FIG. 2C is a graphic representation of GTP levels in A549 cells incubated with the ¹³C-labeled precursors of Adenine (¹³C-A), Guanine (¹³C-G) or with both precursors combined (¹³C-G+¹³C-A), in the presence of ¹⁵N-glutamine. The relative contribution of salvage of ¹³C-G or ¹³C-A vs. de novo synthesis to total GTP levels in each condition is indicated;

FIG. 2D is a graphic representation of ATP levels in A549 cells incubated with the ¹³C-labeled precursors of Adenine (¹³C-A), Guanine (¹³C-G) or with both precursors combined (¹³C-G+¹³C-A), in the presence of ¹⁵N-glutamine. The relative contribution of salvage of ¹³C-G or ¹³C-A vs. de novo synthesis to total ATP levels in each condition is indicated;

FIG. 2E is a graphic representation of proliferation rates of A549 cells treated with the nucleotide precursors guanine (G) with or without adenine (A);

FIG. 2F is a graphic representation of proliferation rates of A549 cells treated with thymidine (T) with or without cytidine (C) or deoxycytidine (dC);

FIG. 3A is a graphic representation of the GDP levels in untreated A549 cells (none), or cells treated with guanine (G) with or without adenine (A);

FIG. 3B is a graphic representation of the GMP levels in untreated A549 cells (none), or cells treated with guanine (G) with or without adenine (A);

FIG. 3C is a graphic representation of the ADP levels in untreated A549 cells (none), or cells treated with guanine (G) with or without adenine (A);

FIG. 3D is a graphic representation of the AMP levels in untreated A549 cells (none), or cells treated with guanine (G) with or without adenine (A);

FIG. 3E is a graphic representation of the GDP levels in A549 cells from ¹³C tracing experiments;

FIG. 3F is a graphic representation of the GMP levels in A549 cells from ¹³C tracing experiments;

FIG. 3G is a graphic representation of the ADP levels in A549 cells from ¹³C tracing experiments;

FIG. 3H is a graphic representation of the AMP levels in A549 cells from ¹³C tracing experiments;

FIG. 3I depicts a graphic representation of the proliferation rate of 143B cells in the presence of guanine (G), adenine (A), or the combination of guanine and adenine (G+A).

FIG. 3J depicts a graphic representation of the proliferation rate of H1299 cells in the presence of guanine (G), adenine (A), or the combination of guanine and adenine (G+A).

FIG. 3K depicts a graphic representation of the proliferation rate of AL1376 cells in the presence of guanine (G), adenine (A, or the combination of guanine and adenine (G+A);

FIG. 4A is a graphic representation of the viability and differentiation (expression of differentiation markers GFP+/CD11b+ and CD11b+ total) of 63.3 cells after treatment with different concentrations of thymidine;

FIG. 4B is a graphic representation of the viability and differentiation (expression of differentiation markers CD11b+/CD16+ and CD11b+ total) of THP1 cells after treatment with different concentrations of thymidine; and

FIG. 4C is a graphic representation of the viability and differentiation (expression of differentiation markers CD11b+/CD16+ and CD11b+ total) of U937 cells after treatment with different concentrations of thymidine.

### DESCRIPTION

The disclosures of these patents, patent applications, and publications in their entireties are hereby incorporated by reference into this application in order to more fully describe the state of the art as known to those skilled therein as of the date of the invention described and claimed herein. The instant disclosure will govern in the instance that there is any inconsistency between the patents, patent applications, and publications and this disclosure.

Unless defined otherwise, all technical and scientific terms used herein have the same meaning as commonly understood by one of ordinary skill in the art to which this disclosure belongs. The initial definition provided for a group or term herein applies to that group or term throughout the present specification individually or as part of another group, unless otherwise indicated.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

The term “treat,” “treated,” “treating,” or “treatment” includes the diminishment or alleviation of at least one symptom associated or caused by the state, disorder or disease being treated. In certain embodiments, the treatment comprises bringing into contact with an infection an effective amount of an anti-infective formulation of the disclosure for conditions related to infections.

As used herein, the term “patient,” “individual,” or “subject” refers to a human or a non-human mammal. Non-human mammals include, but are not limited to, livestock and pets, such as ovine, bovine, porcine, canine, feline, lupine, murine, and marine mammals.

As used herein, the terms “effective amount,” “pharmaceutically effective amount,” and “therapeutically effective amount” refer to a nontoxic but sufficient amount of the purine, purine precursor, purine analog, pyrimidine, pyrimidine precursor, and/or pyrimidine analog to provide the desired biological result. That result may be reduction or alleviation of the signs, symptoms, or causes of a disease, e.g., cancer, or any other desired alteration of a biological system. An appropriate therapeutic amount in any individual case may be determined by one of ordinary skill in the art using routine experimentation.

The term “comprising” encompasses the term “including.”

The term “salvage” as used herein encompasses the use of pre-formed purines or pyrimidines bases in a cell for the synthesis of new nucleosides and nucleotides. Pre-formed purine and pyrimidine bases can be result from RNA or DNA degradation in a cell. The liberated pre-formed purine or pyrimidine bases can subsequently be used to form the new nucleotides. The purine base adenine can be used, for example, to form adenosine mono phosphate (AMP), and the purine base guanine can be used to form guanosine mono phosphate (GMP). Likewise, the pyrimidine base uridine can be used to form uridine monophosphate (UMP), the pyrimidine base cytosine can be used to form cytosine monophosphate (CMP), and the pyrimidine base thymine can be used to form deoxythymidine monophosphate (dTMP). The biosynthetic pathways that utilize the pre-formed bases are called salvage pathways.

In order to divide, cells must double their mass over the course of the cell cycle (Zhu et al. (2019), Nat. Rev. Mol. Cell Biol. 20: 436-450; Vander Heiden et al. (2017) Cell 168: 657-669). As a result, proliferative metabolism is adapted to allow synthesis of the proteins, lipids, and nucleic acids needed to produce two daughter cells. Limiting environmental nutrients or disrupting anabolic reactions can therefore inhibit proliferation, and cells have evolved signaling networks to match growth with metabolic capacity.

Growth control pathways sense metabolic stress and respond to the availability of nutrients needed for biosynthesis. The majority of cell biomass is derived from amino acids (Hosios et al. (2016), Dev. Cell 36: 540-549), and several major growth control pathways sense amino acids and coordinate their use in protein production (Zhu et al. (2019), Nat. Rev. Mol. Cell Biol. 20: 436-450). Depletion of any amino acid resulting in accumulation of uncharged tRNAs leads to activation of general control nonderepressible 2 (GCN2) kinase, which signals to attenuate global translation and promote the synthesis and uptake of amino acids (PMID: 24732012)

In addition to replicating the genome, all proliferating cells also require nucleotides to support increased rRNA and mRNA production. Each nucleotide species has distinct roles in cell metabolism. In addition, levels of each nucleotide vary over a wide range of intracellular concentrations, and will be differentially affected by environmental conditions (Lane et al. (2015) Nucl. Acids Res. 43: 2466-2485). Complex allosteric mechanisms regulate enzymes involved in nucleotide synthesis as one way to promote appropriate balance among nucleotide pools.

In a cancer cell, there are certain internal or endogenous baseline levels of nucleotides. The baseline nucleotide ratio in a cancer cell is the ratio of purine and pyrimidine nucleotides present in the cell without any exogenous intervention. The nucleotide ratio can be defined, for example, as a purine to pyrimidine, or the purine:pyrimidine ratio in the cell. Alternatively, the baseline ratio can be defined as the pyrimidine:purine ratio, or a ratio of specific nucleotides, for instance, the adenine:thymine ratio, or the adenine:guanine ratio.

The baseline nucleotide ratio in a cancer cell is changed by exposing the cell to an agent or condition that directly or indirectly affects the level of a nucleotide in the cell. As provided herein, the purine:pyrimidine ratio or the ratio between individual nucleotides, i.e. the adenine to thymidine, or the adenine to guanine ratio in a cell can be changed in several ways. One way is to add to a cell one or more purine or pyrimidine nucleotides, i.e., by contacting the cell with adenine, guanine, cytidine, thymidine, or with chemical degradation products of the nucleotide such as Xanthine), or a nucleotide precursor, to a cell. Alternatively, the nucleotide ratio in a cell may be altered by adding to/contacting the cell an analog of any of the purine or pyrimidine nucleotides that do not function as inhibitors of pyrimidine or purine biosynthesis.

Purine or pyrimidine bases, nucleosides or nucleotides, or their precursors or analogs may be provided to the cell such that they enter the cell via various mechanisms. For example, the nucleotide, nucleotide precursors, and/or nucleotide analogs may be taken up by the cell in a receptor-independent fashion, e.g., via active of passive endocytosis. Nucleotides, nucleotide precursors, and/or nucleotide analogs can also be internalized via clathrin-mediated endocytosis or macropinocytosis, and then transported to the endosomes, lysosomes, endoplasmic reticulum, Golgi apparatus. Alternatively, the nucleotides, precursors, or analogs can be transported to the inside of the cell via specific receptors. Examples of nucleoside transporters are hCNT1, hCNT2, hCNT3, hENT1, hENT2 and hENT3.

Regardless of the uptake mechanism, by causing a change in the internal baseline ratio, an intra-purine and intra-pyrimidine imbalance results, and such an imbalance leads to replication stress, inhibits cell proliferation in rapidly diving cells such as cancer cells, and elicits differentiation. As described herein, cancer can be treated by creating such nucleotide imbalances.

### I. Inhibition of Cancer Cell Proliferation

The disclosure provides a method of inhibiting cell proliferation in a cancer cell. In this method, the cancer cell is contacted with an agent comprising a purine, a purine precursor, a purine analog that is not an inhibitor of purine biosynthesis, a pyrimidine, a pyrimidine precursor, or a pyrimidine analog that is not an inhibitor of pyrimidine biosynthesis, in amount sufficient to change the internal baseline ratio of purine:pyrimidine in the cancer cell, thereby resulting in a nucleotide imbalance. The resulting nucleotide imbalance inhibits the cancer cell from proliferating.

The contacting purine can be, for example, Adenine or Guanine or a precursor or analog of the foregoing purines. Exemplary purine precursors include, but are not limited to, AIR, CAIR, SACAIR, AICAR, FAICAR inosine monophosphate (IMP), adenylosuccinate, xanthine, and hypoxanthine. A purine analog is any purine analog that is not an inhibitor of purine biosynthesis. Exemplary purine analogs include, but are not limited to Adenine, Guanine or any of the listed precursors, with one or more substitutions, that do not directly interfere with purine biosynthesis, and 8-amino-adenosine.

The contacting pyrimidine can be, for example, Cytosine, Thymine or Uracil, or a precursor or analog (that does not inhibit pyrimidine biosynthesis) of the foregoing pyrimidines. Exemplary pyrimidine precursors include, but are not limited to, dihydroorotate, orotate, uracil monophosphate (UMP), UDP, CMP, or CDP. A pyrimidine analog is any pyrimidine analog that is not an inhibitor of pyrimidine biosynthesis. Exemplary pyrimidine analogs include, but are not limited to substituted Cytosine, Thymine, Uracil, or any of the listed precursors (with one or more substitutions) that do not interfere with pyrimidine biosynthesis, cytarabine, nalarabine, sapacitabine, and ARC (4-amino-6-hydrazino-7-β-D-ribofuranosyl-7H-pyrrolo[2,3-d]-pyrimidine-5-carboxamide).

The nucleotide ratio in a cancer cell can also be altered by contacting it with a pyrimidine or a purine base, nucleoside or nucleotide and with one or more inhibitors of the purine or pyrimidine synthesis pathways, respectively. Inhibitors can be purine or pyrimidine analogs, or inhibitors of critical enzymes in the purine and pyrimidine metabolic pathways.

The nucleotide ratio in a cancer cell can also be altered by contacting it with pyrimidine bases, nucleosides or nucleotides and with purine analogs that inhibit purine biosynthesis. Exemplary purine analogs that inhibit purine biosynthesis are Azathioprine, Mercaptopurine, Clofarabine, Thioguanine, Fludarabine, Pentostatin, Cladribine, 8-amino-adenosine, or Acycloguanosine. Alternatively, pyrimidine bases, nucleosides or nucleotides can be combined with agents that inhibit enzymes critical in purine metabolism. Exemplary inhibitors of the enzyme IMPDH include, but are not limited to Mercaptopurine, 6-thioguanine, Mycophenolic acid, MMF, and Mizoribine.

Alternatively, the nucleotide ratio in a cancer cell can also be altered by contacting it with purine bases, nucleosides or nucleotides and with pyrimidine analogs that inhibit pyrimidine biosynthesis. Exemplary pyrimidine analogs that inhibit pyrimidine biosynthesis are 5-Fluorouracil (thymidilate synthesis inhibitor), Floxuridine, Cytarabine, 6-azauracil, Gemcitabine, Idoxuridine, 3′-azido-3′deoxythymidine, RX-3117 (flurocyclopentenylcytosine), Pyrazofurin, cytarabine, nalarabine, sapacitabine, and ARC (4-amino-6-hydrazino-7-β-D-ribofuranosyl-7H-pyrrolo[2,3-d]-pyrimidine-5-carboxamide). Alternatively, purine bases, nucleosides or nucleotides can be combined with agents that inhibit enzymes critical in pyrimidine metabolism. Exemplary inhibitors of the enzyme DHODH include, but are not limited to Brequinar, Teriflunomide, Leflunomide, and Lapachol; inhibitors of CTP synthase include, but are not limited to Cyclopentenyl cytosine; inhibitors of UMPS include, but are not limited to 6-azauridine; inhibitors of OMPDC include; but are not limited to Pyrazofurin; and inhibitors of DHFR include, but are not limited to Pyrimethamine and Methotrexate.

Further, the nucleotide ratio in a cancer cell can also be altered by contacting it with purine or pyrimidine bases, nucleosides or nucleotides, precursors thereof, analogs thereof which are not biosynthesis inhibitors, together with inhibitor of either the purine or pyrimidine synthesis pathways. Exemplary inhibitors that target GARFT include, but are not limited to the antifolate lometrexol.

For example, to change the internal ratio of purine:pyrimidines in the cell, the cell can be contacted with a nucleoside, comprising a purine. Examples of such nucleosides are Adenosine comprising Adenine, Deoxyadenosine comprising adenine, Guanosine comprising Guanine, or Deoxyguanosine comprising Guanine. Alternatively, the cell can be contacted with a nucleotide comprising a nucleoside comprising a purine. Exemplary nucleotides are Adenylate comprising Adenosine, Deoxyadenylate comprising Deoxyadenosine, Guanylate comprising Guanosine, or Deoxyguanylate comprising Deoxyguanosine. In another example, the cell is contacted with a nucleoside comprising a pyrimidine. Examples of such nucleosides are Cytidine comprising Cytosine, Deoxycytidine comprising Cytosine, Thymidine comprising Thymine, Deoxythymidine comprising Thymine, or Uracil comprising Uridine. In yet another example, the cell is contacted with a nucleotide comprising a nucleoside comprising a pyrimidine. Exemplary nucleotides are Cytidylate comprising Cytidine, Deoxycytidylate comprising Deoxycytidine, Thymidylate comprising Thymidine, Deoxythymidinylate comprising Deoxythymidine, or Uridylate comprising Uridine.

The amount of purine, purine precursor, purine analog, pyrimidine, pyrimidine precursor, and/or pyrimidine analog added to the cancer cell is that amount which, once entering the cell, changes the cell's internal baseline nucleotide ratio and inhibits proliferation in the cell. The reduction in proliferation can be measured by any method known in the art, measuring a cell's proliferation rate. The proliferation rate can be measured, e.g., by determining the overall metabolic activity in a cell. An exemplary method for determining metabolic activity is by employing dyes that permeabilize the cell and react with certain metabolic enzymes or metabolic products. Detection of the colored product can be performed by methods including, but not limited to ELISA, and Flow cytometry. An exemplary dye that measures metabolic cell activity is MTT, which produces a purple color in proliferating cells. Another exemplary dye is WST-1, which likewise produces a colored product. Alternatively, cell metabolic activity can be determined with fluorescent dyes, that in general have a greater sensitivity than colorimetric dyes. Further, cell metabolic activity can be measured with the BrdU (5′-Bromo-2′Deoxyuridine) incorporation assay. BrdU is a thymidine analog that is incorporated in proliferating cells. It can be detected with an anti-BrdU antibody and a labeled (i.e., fluorescently labeled) secondary antibody.

The cancer cell may be a liquid cancer cell. Liquid cancers are cancers of the cells in the circulatory system. Examples of liquid cancers are, but are not limited to Acute lymphoblastic leukemia (ALL), Chronic lymphocytic leukemia (CLL), Acute myelogenous leukemia (AML), Chronic myelogenous leukemia (CML), Hairy cell leukemia (HCL), T-cell prolymphocytic leukemia (T-PLL), Large granular lymphocytic leukemia, Adult T-cell leukemia, and Clonal eosinophilias. The cancer cell may be in a mammalian species such as, but not limited to a human, monkey, dog, cat, cow, pig, horse, rabbit, rat, or a mouse. Exemplary liquid cancer cell lines include, but are not limited to, the human leukemia cancer cell lines THP-1 and U937.

The cancer cell may also be a solid cancer cell. Solid cancer cells may originate in any of the following organs including, but not limited to, bone and muscle, brain, eye, breast, endocrine system including the thyroid; genitourinary tract including kidneys, ovaries, penis, prostate, testicles, urethra, vagina, head and neck including esophagus, nasopharynx, tongue, salivary gland, skin, and the thorax and respiratory tract including lungs, larynx. Exemplary solid cancer cell lines include, but are not limited to, the adenocarcinomic human alveolar basal epithelial cell line A549, the osteosarcoma cell line 143B, the human non-small cell lung carcinoma cell line H1299, the human ovarian cancer cell line A2780, the osteosarcoma cell line U205, and the breast cancer cell line MDA-MB-468.

Disrupting pro-growth signaling pathways, limiting amino acid availability, blocking mitochondrial respiration, and inhibiting nucleotide synthesis all can inhibit cell proliferation. Inhibiting production of either purines using lometrexol (LTX) or pyrimidines using brequinar (BRQ) depletes total purine or pyrimidine levels and also blocks proliferation.

Other perturbations to nucleotide homeostasis were investigated for their detrimental effect on dividing cells, contacting cells with individual nucleobases and nucleosides, which can be salvaged to produce nucleotides.

Single nucleotide supplementation was found to impair proliferation (TABLE 1), which shows doses of adenosine (A), deoxyadenosine (dA), thymidine (T), or guanine (G), added to various cell lines, leading to 50% inhibition of proliferation of the indicated cell lines. This effect was dose-titratable over a 0.1 mM to 2.0 mM range, with increasing concentrations of multiple single nucleotides completely preventing cell proliferation.

TABLE 1

Added nucleoside/base (mM)

| Cell line | A | dA | T | G |
|---|---|---|---|---|
| A549 | 2.1 | 1.2 | 0.5 | 0.08 |
| 143B | 1 | 0.6 | NA | 0.12 |
| H1299 | 1.1 | 0.8 | 0.7 | 0.2 |
| A2780 | 1 | 0.3 | 0.3 | 0.1 |
| U2OS | 0.95 | 0.6 | 0.5 | 0.25 |
| MDA-MB-468 | 0.8 | 0.225 | 0.15 | 0.15 |

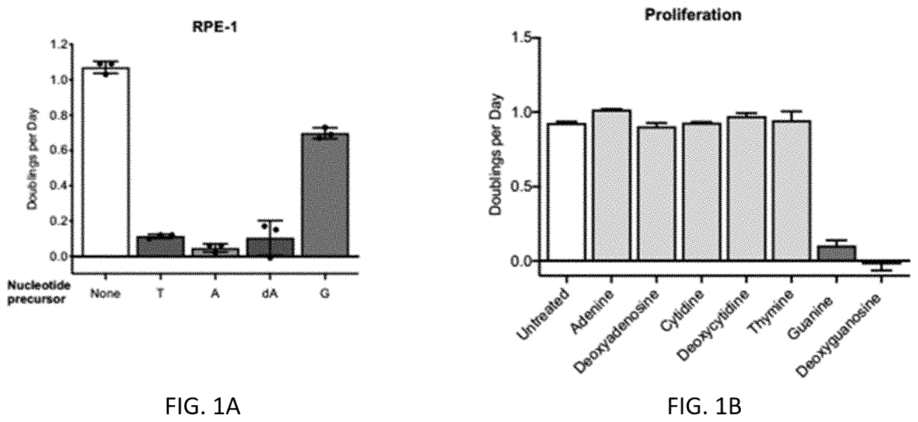

In Table 1, the approximate concentration of each nucleotide that when added slows the proliferation of the indicated cells by half is shown. These data indicated how different cancer cells respond differently to different concentration of individual nucleotides. Deoxycytidine blocked proliferation at higher concentrations (FIG. 1B).

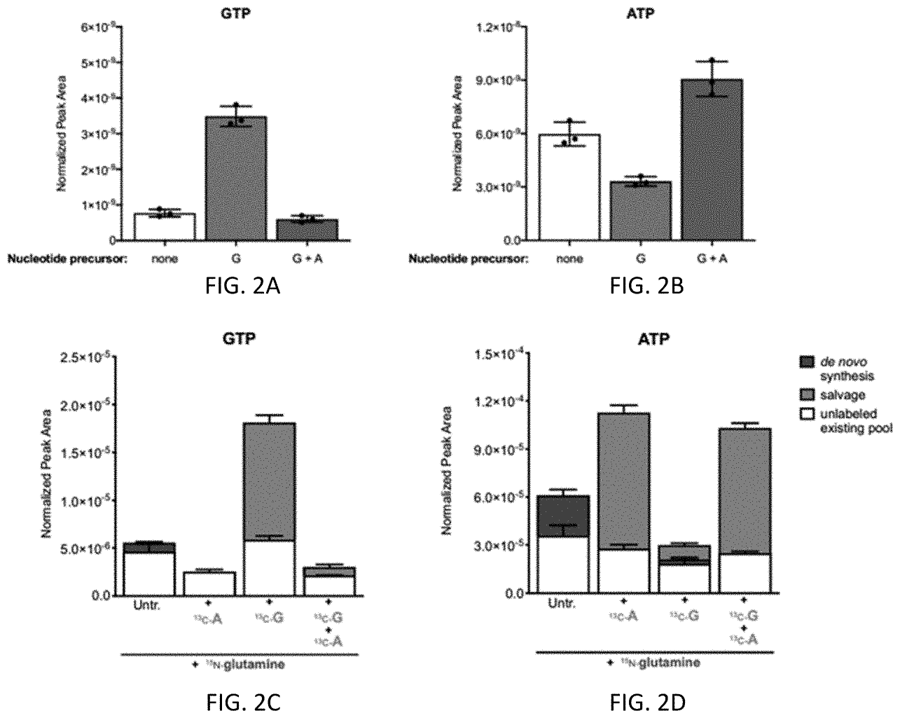

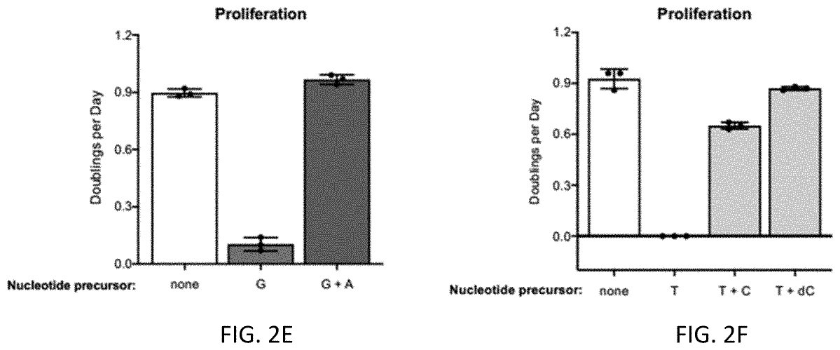

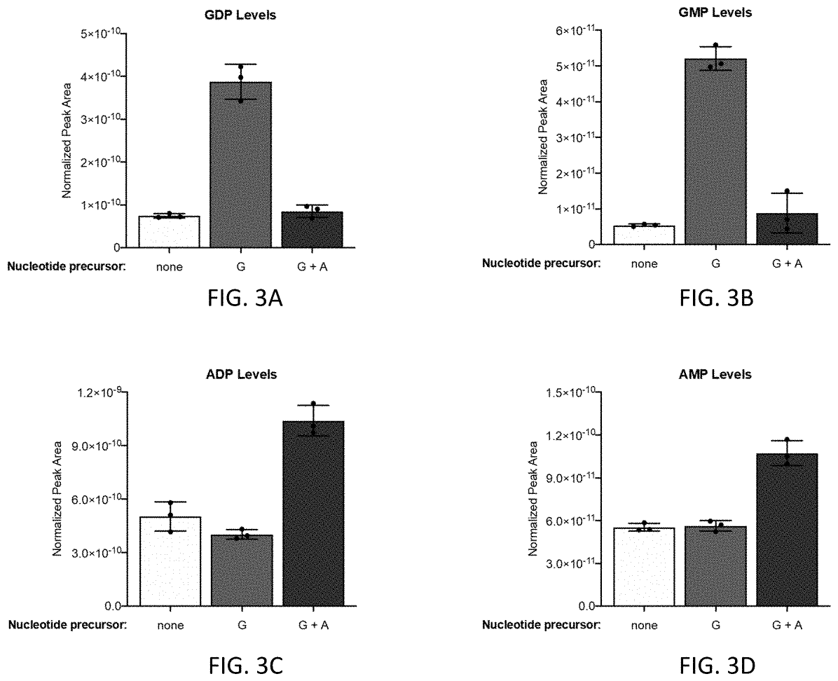

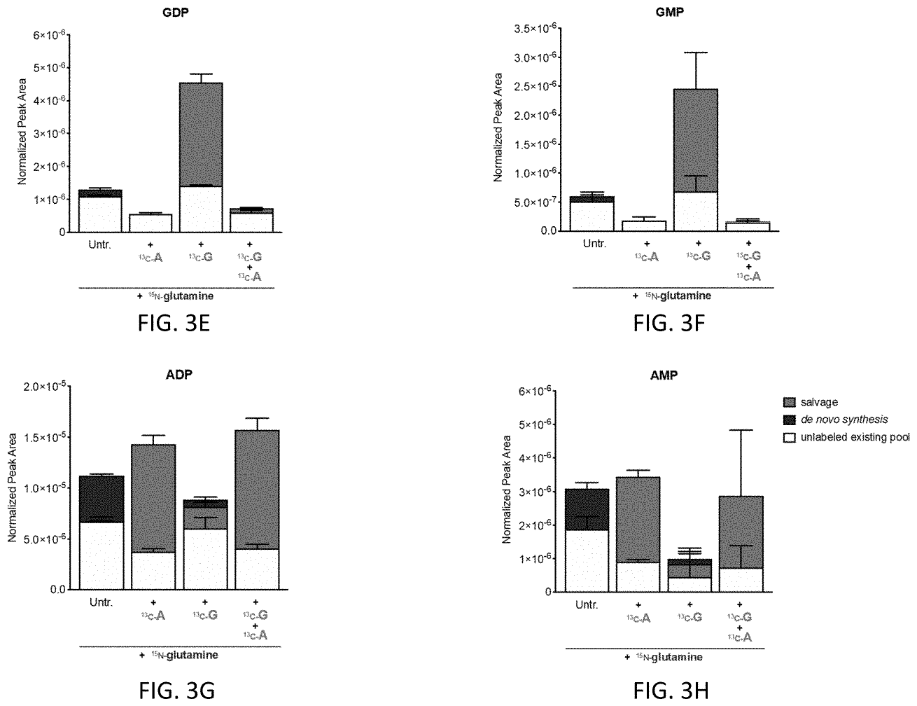

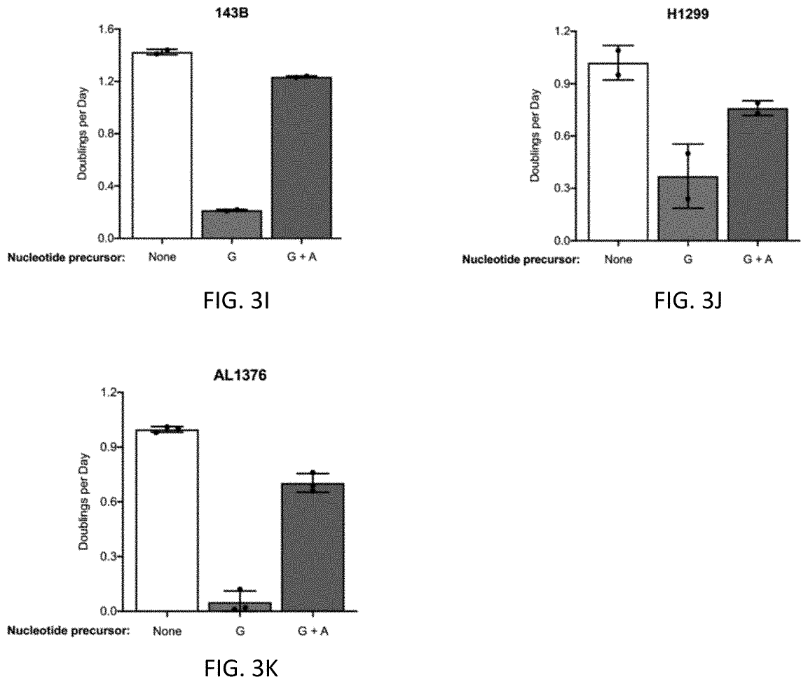

As shown in FIGS. 2A and 3A-3B, Guanine supplementation dramatically increased intracellular pools of guanylate nucleotides (GTP/GDP/GMP) in A549 cells. Guanine also decreased intracellular levels of adenylate nucleotides (ATP/ADP/AMP) (FIGS. 2B and 3C-3D). These data show that guanine salvage disrupts normal intra-purine balance by increasing the ratio of guanylate (G) to adenylate (A) nucleotides. Providing exogenous adenine together with guanine restored the baseline balance of intracellular G and A nucleotides (FIG. 2A-2B).

To show how providing guanine depletes intracellular A nucleotides, stable isotope tracing was done to determine the contribution of both salvage and de novo synthesis to cellular purines. To assess de novo synthesis, incorporation of amide-¹⁵N-glutamine into purines was measured. The amide nitrogen of glutamine is incorporated during AMP and GMP synthesis such that purines labeled with ¹⁵N have been made de novo. ¹³C-guanine and ¹³C-adenine were also used to measure how salvage of these nucleobases contributes to intracellular purines. ¹³C-labeled purines are derived from the addition of guanine or adenine to a ribose-phosphate backbone. A subset of purine nucleotides in untreated cells were labeled with ¹⁵N, reflecting their production via de novo synthesis in comparison to those nucleotides that were labeled with ¹³C that were salvaged from the environment (FIGS. 2C-2D, and 3E-3H).

Providing ¹³C-adenine increased levels of A nucleotides, the majority of which were ¹³C-labeled and therefore derived from adenine salvage. Similarly, salvage of 13C-guanine accounted for the increase in G nucleotide levels upon guanine supplementation. Moreover, providing either ¹³C-adenine or ¹³C-guanine eliminated the contribution of de novo synthesis to both A and G nucleotide pools (FIGS. 2C-2D, and 3E-3H). Aberrantly high levels of G nucleotides derived from guanine salvage may inhibit de novo synthesis of both G and A nucleotides, resulting in depletion of A nucleotides. Thus, exogenous G disrupts intra-purine balance by increasing levels of G nucleotides while preventing the synthesis of A nucleotides.

Providing adenine together with guanine restored proliferation across multiple cell types (FIGS. 3I-3K), indicating that purine imbalance with excess G nucleotides accounts for decreased proliferation. To assess whether an analogous imbalance in relative nucleotide levels provides a general explanation for why excess levels of a single nucleotide precursor impairs, intracellular nucleotide levels upon addition of A-, dA-, G-, dG-, T- and C-precursors were compared, each at concentrations that inhibit proliferation. Each salvage precursor altered relative nucleotide levels in different ways (FIG. 2E). Accordingly, providing cytidylate (C) precursors to balance pyrimidine levels restored proliferation of cells treated with thymidylate (T) nucleotides (FIG. 2F). Together, these data show that both pyrimidine and purine imbalances can lead to inhibition of proliferation.

### II. Induction of Cancer Cell Differentiation

The disclosure provides a method of inducing differentiation in a cancer cell. In this method, the cancer cell is contacted with an agent comprising a purine, a purine precursor, a purine analog that is not an inhibitor of purine biosynthesis, a pyrimidine, a pyrimidine precursor, or a pyrimidine analog that is not an inhibitor of pyrimidine biosynthesis, in amount sufficient to change the internal baseline ratio of purine:pyrimidine in the cancer cell, thereby resulting in a nucleotide imbalance. The resulting nucleotide imbalance induces the cancer cell to differentiate

The contacting purine can be, for example, Adenine or Guanine or a precursor or analog of the foregoing purines. Exemplary purine precursors include, but are not limited to, AIR, CAIR, SACAIR, AICAR, FAICAR inosine mono phosphate (IMP), adenylosuccinate, xanthine, and hypoxanthine. A purine analog is any purine analog that is not an inhibitor of purine biosynthesis. Exemplary purine analogs include, but are not limited to Adenine, Guanine or any of the listed precursors, with one or more substitutions, that do not interfere with purine biosynthesis, and 8-amino-adenosine.

The contacting pyrimidine can be, for example, Cytosine, Thymine or Uracil, or an analog or precursor of the foregoing pyrimidines. Exemplary pyrimidine precursors include, but are not limited to dihydroorotate, orotate, uracil monophosphate (UMP), UDP, CMP, or CDP. A pyrimidine analog is any pyrimidine analog that is not an inhibitor of pyrimidine biosynthesis Exemplary pyrimidine analogs include, but are not limited to Cytosine, Thymine, Uracil, or any of the listed precursors, with one or more substitutions that do not interfere with pyrimidine biosynthesis, cytarabine, nalarabine, sapacitabine, and ARC (4-amino-6-hydrazino-7-β-D-ribofuranosyl-7H-pyrrolo[2,3-d]-pyrimidine-5-carboxamide).

The nucleotide ratio in a cancer cell can also be altered by contacting it with a pyrimidine or a purine base, nucleoside or nucleotide and with one or more inhibitors of the purine or pyrimidine synthesis pathways, respectively. Inhibitors can be purine or pyrimidine analogs, or inhibitors of critical enzymes in the purine and pyrimidine metabolic pathway, as described above.

The nucleotide ratio in a cancer cell can also be altered by contacting it with Pyrimidine bases, nucleosides or nucleotides and with purine analogs that inhibit purine biosynthesis. Exemplary purine analogs that inhibit purine biosynthesis are Azathioprine, Mercaptopurine, Clofarabine, Thioguanine, Fludarabine, Pentostatin, Cladribine or Acycloguanosine. Alternatively, pyrimidine bases, nucleosides or nucleotides can be combined with agents that inhibit enzymes critical in purine metabolism. Exemplary inhibitors of the enzyme IMPDH include, but are not limited to, Mercaptopurine, 6-thioguanine, Mycophenolic acid, MMF, and Mizoribine.

The nucleotide ratio in a cancer cell can also be altered by contacting it with Purine bases, nucleosides or nucleotides and with pyrimidine analogs that inhibit pyrimidine biosynthesis. Exemplary pyrimidine analogs that inhibit pyrimidine biosynthesis are 5-Fluorouracil (thymidylate synthesis inhibitor), Floxuridine, Cytarabine, 6-azauracil, Gemcitabine, Idoxuridine, 3′-azido-3′deoxythymidine, RX-3117 (flurocyclopentenylcytosine), Pyrazofurin, cytarabine, nalarabine, sapacitabine, and ARC (4-amino-6-hydrazino-7-β-D-ribofuranosyl-7H-pyrrolo[2,3-d]-pyrimidine-5-carboxamide). Alternatively, purine bases, nucleosides or nucleotides can be combined with agents that inhibit enzymes critical in pyrimidine metabolism. Exemplary inhibitors of the enzyme DHODH include, but are not limited to Brequinar, Teriflunomide, Leflunomide, Lapachol; inhibitors of CTP synthase include, but are not limited to, Cyclopentenyl cytosine; inhibitors of UMPS include, but are not limited to 6-azauridine; inhibitors of OMPDC include, but are not limited to Pyrazofurin; and inhibitors of DHFR include, but are not limited to Pyrimethamine and Methotrexate.

Further, the nucleotide ratio in a cancer cell can also be altered by contacting it with purine or pyrimidine bases, nucleosides or nucleotides and with an inhibitor that affects both the purine and pyrimidine synthesis pathways. Exemplary inhibitors that target GARFT include, but are not limited to the antifolate lometrexol.

To change the internal baseline ratio of purine:pyrimidine in the cancer cell, for example, the cell can be contacted with a nucleoside, comprising a purine. Examples of such nucleosides are Adenosine comprising Adenine, Deoxyadenosine comprising adenine, Guanosine comprising Guanine, or Deoxyguanosine comprising Guanine. Alternatively, the cell can be contacted with a nucleotide comprising a nucleoside comprising a purine.

Exemplary nucleotides are Adenylate comprising Adenosine, Deoxyadenylate comprising Deoxyadenosine, Guanylate comprising Guanosine, or Deoxyguanylate comprising Deoxyguanosine. In another example, the cell is contacted with a nucleoside comprising a pyrimidine. Examples of such nucleosides are Cytidine comprising Cytosine, Deoxycytidine comprising Cytosine, Thymidine comprising Thymine, Deoxythymidine comprising Thymine, or Uracil comprising Uridine. In yet another example, the cell is contacted with a nucleotide comprising a nucleoside comprising a pyrimidine. Exemplary nucleotides are Cytidylate comprising Cytidine, Deoxycytidylate comprising Deoxycytidine, Thymidylate comprising Thymidine, Deoxythymidinylate comprising Deoxythymidine, or Uridylate comprising Uridine.

The amount of purine, purine precursor, purine analog, pyrimidine, pyrimidine precursor, and/or pyrimidine analog added to the cancer cell is that amount which, once entering the cell, changes the cell's internal baseline nucleotide ratio and promotes differentiation in the cell. Cell differentiation can be measured by any method known in the art, e.g., by measuring the expression of differentiation markers. Differentiation can be measured by measuring expression of markers for differentiation of AML or other myeloid malignancies (e.g., MPO, ELANE, CTSG, LTF, LCN2, CAMP, MMP8, MMP9, S100A8 or RNA markers GFI1, SPI1, CEBPE, CEBPA, IRF8). Expression can be measured by any method known in the art, i.e. at the RNA level by performing qPCR or RNAseq, or at the protein level by Western blotting or flow cytometry using specific antibodies. Flow cytometry can for example be used, for example, to track changes in the expression of CD11b, CD13, CD14, CD15, CD16, Ly6G, and CD34, CD33, CD38, CD45, CD19, CD3, CD117, CD235A, Ly6c2, CD115, or Vcam1.

The cancer cell may be a liquid cancer cell. Liquid cancers are cancers of the cells in the circulatory system. Examples of liquid cancers are, but are not limited to Acute lymphoblastic leukemia (ALL), Chronic lymphocytic leukemia (CLL), Acute myelogenous leukemia (AML), Chronic myelogenous leukemia (CML), Hairy cell leukemia (HCL), T-cell prolymphocytic leukemia (T-PLL), Large granular lymphocytic leukemia, Adult T-cell leukemia, and Clonal eosinophilias. The cancer cell may be in a mammalian species such as, but not limited to a human, monkey, dog, cat, cow, pig, horse, rabbit, rat, or a mouse. Exemplary liquid cancer cell lines include, but are not limited to the human leukemia cancer cell lines THP-1 and U937.

The cancer cell may also be a solid cancer cell. Solid cancer cells may originate in any of the following organs including, but not limited to bone and muscle; brain; eye, breast; endocrine system including the thyroid; genitourinary tract including kidneys, ovaries, penis, prostate, testicles, urethra, vagina; head and neck including esophagus, nasopharynx, tongue, salivary gland; skin; and the thorax and respiratory tract including lungs, larynx. Exemplary solid cancer cell lines include, but are not limited to the adenocarcinomic human alveolar basal epithelial cell line A549, the osteosarcoma cell line 143B, the human non-small cell lung carcinoma cell line H1299, the human ovarian cancer cell line A2780, the osteosarcoma cell line U205, and the breast cancer cell line MDA-MB-468.

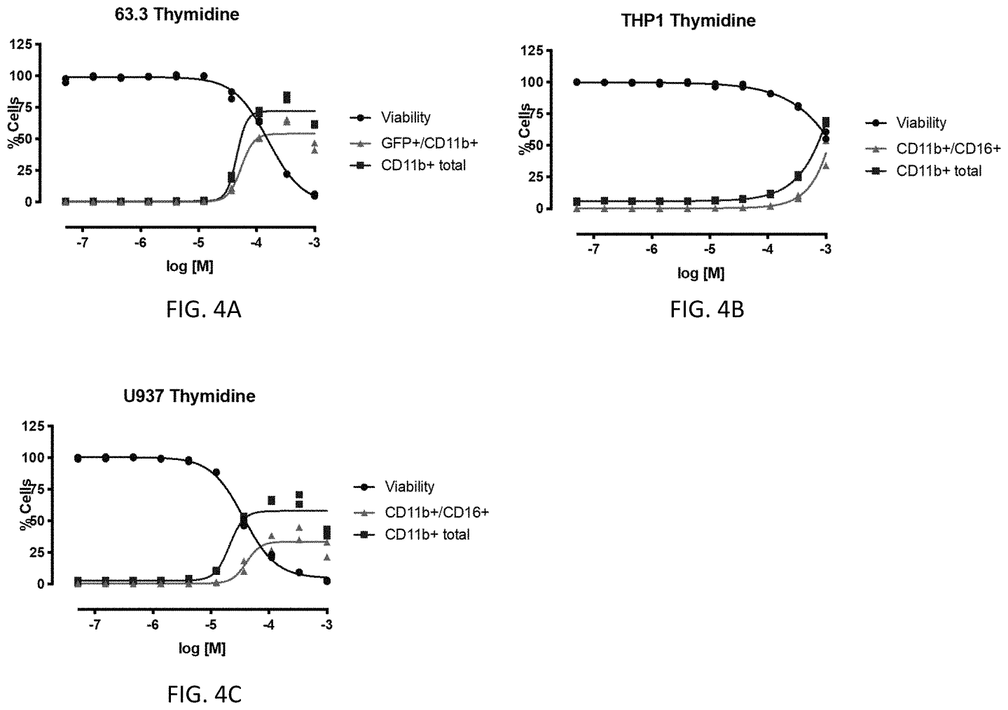

That exogenous nucleotides can direct cancer cells to undergo differentiation was determined as follows. Thymidine was added at concentrations ranging from 10⁻³ to 10⁻⁷ M to 63.3 cells (FIG. 4A), THP1 cells (FIG. 4B) and U937 cells (FIG. 4C). Cell differentiation was monitored by CD11b+/CD16+ expression, and by CD11b+ total expression. In all three cell lines, the addition of thymidine induced cell differentiation in a concentration dependent manner. Similar effects were observed with Adenine at higher concentrations.

### III. Induction of Replication Stress in a Cancer Cell

The disclosure also provides a method of inducing replication stress in a cancer call. In this method, the cancer cell is contacted with an agent comprising a purine, a purine precursor, purine analog that is not an inhibitor of purine biosynthesis, a pyrimidine, a pyrimidine precursor, or a pyrimidine analog that is not an inhibitor of pyrimidine biosynthesis, in amount sufficient to change the internal baseline ratio of purine:pyrimidine in the cancer cell, thereby resulting in a nucleotide imbalance. The resulting nucleotide imbalance causes replication stress in a cancer cell.

Replication stress in the cancer cell can be induced by contacting the cell with a purine, for example, Adenine or Guanine or a precursor or analog of the foregoing purines. Exemplary purine precursors include, but are not limited to, AIR, CAIR, SACAIR, AICAR, FAICAR inosine mono phosphate (IMP), adenylosuccinate, xanthine, and hypoxanthine. A purine analog is any purine analog that is not an inhibitor of purine biosynthesis. Exemplary purine analogs include, but are not limited to, substituted Adenine, Guanine or any of the listed precursors (with one or more substitutions), that do not interfere with purine biosynthesis, or 8-amino-adenosine.

Replication stress in the cancer cell can also be induced by contacting the cell a pyrimidine, for example, Cytosine, Thymine or Uracil, or an analog or precursor of the foregoing pyrimidines. Exemplary pyrimidine precursors include, but are not limited to, dihydroorotate, orotate, uracil monophosphate (UMP), UDP, CMP, or CDP. A pyrimidine analog is any pyrimidine analog that is not an inhibitor of pyrimidine biosynthesis Exemplary pyrimidine analogs include, but are not limited to, Cytosine, Thymine, Uracil, or any of the listed precursors, with one or more substitutions that do not interfere with pyrimidine biosynthesis, cytarabine, nalarabine, sapacitabine, and ARC (4-amino-6-hydrazino-7-β-D-ribofuranosyl-7H-pyrrolo[2,3-d]-pyrimidine-5-carboxamide).

Replication stress in the cancer cell can be induced by contacting the cell with a pyrimidine or a purine base, nucleoside, or nucleotide, in combination with one or more inhibitors of the purine or pyrimidine synthesis pathways, respectively. Inhibitors can be purine or pyrimidine analogs, or inhibitors of critical enzymes in the purine and pyrimidine metabolic pathways.

Replication stress in the cancer cell can be induced by contacting the cell with Pyrimidine bases, nucleosides, or nucleotides, in combination with purine analogs that inhibit purine biosynthesis. Exemplary purine analogs that inhibit purine biosynthesis include, but are not limited to, Azathioprine, Mercaptopurine, Clofarabine, Thioguanine, Fludarabine, Pentostatin, Cladribine, 8-amino-adenosine, or Acycloguanosine. Alternatively, replication stress in the cancer cell can be induced by contacting the cell with pyrimidine bases, nucleosides, or nucleotides, in combination with agents that inhibit enzymes critical in purine metabolism. Exemplary inhibitors of the enzyme IMPDH include, but are not limited to, Mercaptopurine, 6-thioguanine, Mycophenolic acid, MMF, and Mizoribine.

The nucleotide ratio in a cancer cell can be altered, hence inducing replication stress, in a cancer cell by contacting the cell with purine bases, nucleosides, or nucleotides, precursors thereof, and analogs thereof that are not purine or pyrimidine biosynthesis inhibitors, in combination with pyrimidine analogs that inhibit pyrimidine biosynthesis. Exemplary pyrimidine analogs that inhibit pyrimidine biosynthesis include, but are not limited to, 5-Fluorouracil (thymidilate synthesis inhibitor), Floxuridine, Cytarabine, 6-azauracil, Gemcitabine, Idoxuridine, 3′-azido-3′deoxythymidine, RX-3117 (flurocyclopentenylcytosine), Pyrazofurin, cytarabine, nalarabine, sapacitabine, and ARC (4-amino-6-hydrazino-7-β-D-ribofuranosyl-7H-pyrrolo[2,3-d]-pyrimidine-5-carboxamide). Alternatively, replication stress in the cancer cell can be induced by contacting the cell with purine bases, nucleosides or nucleotide in combination with agents that inhibit enzymes critical in pyrimidine metabolism. Exemplary inhibitors of the enzyme DHODH include, but are not limited to, Brequinar, Teriflunomide, Leflunomide, Lapachol, Thymidilate synthase, and 5FU, inhibitors of CTP synthase include, but are not limited to, Cyclopentenyl cytosine, inhibitors of UMPS include, but are not limited to, 6-azauridine; inhibitors of OMPDC include, but are not limited to Pyrazofurin; and inhibitors of DHFR include, but are not limited to, Pyrimethamine and Methotrexate.

Further, replication stress in the cancer cell can be induced by contacting the cell with purine or pyrimidine bases, nucleosides, or nucleotides, in combination with an inhibitor that affects both the purine and pyrimidine synthesis pathways. Exemplary inhibitors that target GARFT include, but are not limited to, the antifolate, lometrexol.

To change the internal baseline ratio of purine:pyrimidine in the cancer cell, for example, the cell can be contacted with a nucleoside, comprising a purine. Examples of such nucleosides are Adenosine comprising Adenine, Deoxyadenosine comprising adenine, Guanosine comprising Guanine, or Deoxyguanosine comprising Guanine. Alternatively, the cell can be contacted with a nucleotide comprising a nucleoside comprising a purine. Exemplary nucleotides are Adenylate comprising Adenosine, Deoxyadenylate comprising Deoxyadenosine, Guanylate comprising Guanosine, or Deoxyguanylate comprising Deoxyguanosine. In another example, the cell is contacted with a nucleoside comprising a pyrimidine. Examples of such nucleosides are Cytidine comprising Cytosine, Deoxycytidine comprising Cytosine, Thymidine comprising Thymine, Deoxythymidine comprising Thymine, or Uracil comprising Uridine. In yet another example, the cell is contacted with a nucleotide comprising a nucleoside comprising a pyrimidine. Exemplary nucleotides are Cytidylate comprising Cytidine, Deoxycytidylate comprising Deoxycytidine, Thymidylate comprising Thymidine, Deoxythymidinylate comprising Deoxythymidine, or Uridylate comprising Uridine.

The cancer cell is contacted with that amount of purine, purine precursor, purine analog, pyrimidine, pyrimidine precursor, and/or pyrimidine which, once entering the cell, changes the cell's internal baseline purine:pyrimidine ratio and induces replication stress in the cell. The induction of replication stress can be measured, in any way known in the art, e.g., with the Comet assay which measures DNA damage. In this assay, cells are mixed in low melting point agarose on a microscope slide and are lysed with a detergent, e.g., 1% N-lauryl sarcosine, 0.5% Triton X-100, DMSO, and high salt, e.g., 2.5 M NaCl, 0.1 M EDTA and 10 mM Tris, to form nucleoids containing supercoiled loops of DNA that are linked to the nuclear matrix. After electrophoresis at high pH structures can be observed resembling comets, visualized by fluorescence microscopy. The intensity of the comet tail relative to the head is proportional to the number of breaks in the DNA. Comets can be scored with dedicated software such as, but not limited to, CaspLab software or OpenComet software.

The cancer cell may be a liquid cancer cell. Liquid cancers are cancers of the cells in the circulatory system. Examples of liquid cancers are, but are not limited to, Acute lymphoblastic leukemia (ALL), Chronic lymphocytic leukemia (CLL), Acute myelogenous leukemia (AML), Chronic myelogenous leukemia (CML), Hairy cell leukemia (HCL), T-cell prolymphocytic leukemia (T-PLL), Large granular lymphocytic leukemia, Adult T-cell leukemia, and Clonal eosinophilias. The cancer cell may be in a mammalian species such as, but not limited to, a human, monkey, dog, cat, cow, pig, horse, rabbit, rat, or a mouse. Exemplary liquid cancer cell lines include, but are not limited to, the human leukemia cancer cell line U937.

The cancer cell may also be a solid cancer cell. Solid cancer cells may originate in any of the following organs including, but not limited to, bone and muscle; brain; eye, breast; endocrine system including the thyroid; genitourinary tract including kidneys, ovaries, penis, prostate, testicles, urethra, vagina; head and neck including esophagus, nasopharynx, tongue, salivary gland; skin; and the thorax and respiratory tract including lungs, larynx. Exemplary solid cancer cell lines include, but are not limited to, the adenocarcinomic human alveolar basal epithelial cell line A549, the osteosarcoma cell line 143B, the human non-small cell lung carcinoma cell line H1299, the human ovarian cancer cell line A2780, the osteosarcoma cell line U205, and the breast cancer cell line MDA-MB-468.

To determine if nucleotide imbalance causes replication stress in a cancer cell, targets of the ATR and ATM kinases were examined after the addition of a nucleotide to change the internal nucleotide ratio. The ATR and ATM kinases are components of the cellular response to DNA damage, and sense single-stranded DNA and DNA double-strand breaks, respectively. The respective targets of ATR and ATM, Chk1 and Chk2, are major effectors of the DNA damage response. Treatment with exogenous guanine caused robust phosphorylation of both Chk1 and Chk2, with higher concentrations of guanine that inhibit proliferation to a greater extent inducing a stronger signaling response. The appearance of p-Chk1 occurred first between 12 hours and 24 hours of guanine treatment, followed by the appearance of p-Chk2 between 48 hours and 72 hours. Induction of the replication stress response was reversed by addition of adenine together with guanine. A similar induction of ATR and ATM signaling occurred with any excess nucleotide supplementation that impaired S phase progression, but not with leucine deprivation, consistent with impaired DNA replication. Inhibiting total purine or pyrimidine synthesis induced phosphorylation of Chk1 and Chk2 but to a lesser extent than Guanosine treatment.

A small fraction of guanine-treated cells exhibited minor increases in DNA damage at 24 hours as measured by a comet tail assay (Gyori et al., (2014), Redox Biol. 2: 457-465). At that time, the signaling response is already robust, indicating that replication stress-sensing pathways are activated under nucleotide imbalance without large amounts of DNA damage. Further, the finding that metabolic regulatory mechanisms fail to halt cell growth and prevent S phase entry downstream of nucleotide imbalance demonstrates that replication stress-sensing constitutes the major signaling response to nucleotide imbalance.

### IV. Treatment of Cancer

The disclosure also provides a method of treating a cancer in a patient. In this method, an agent is administered to the patient in amount sufficient to change the internal baseline ratio of purine:pyrimidine in the cancer. The agent comprises a purine, a purine precursor, a purine analog that is not an inhibitor of purine biosynthesis, a pyrimidine, a pyrimidine precursor, or a pyrimidine analog that is not an inhibitor of pyrimidine biosynthesis. The resulting change in the internal baseline ratio resulting in a nucleotide imbalance, thereby treating the cancer by inducing replication stress in, inhibiting proliferation of, and/or inducing differentiation of, the cancer.

The administered purine can be, for example, Adenine or Guanine or a precursor or analog of the foregoing purines. Exemplary purine precursors include, but are not limited to, AIR, CAIR, SACAIR, AICAR, FAICAR inosine monophosphate (IMP), adenylosuccinate, xanthine, and hypoxanthine. A purine analog is any purine analog that is not an inhibitor of purine biosynthesis. Exemplary purine analogs include, but are not limited to Adenine, Guanine or any of the listed precursors, with one or more substitutions, that do not interfere with purine biosynthesis, or 8-amino-adenosine.

The administered pyrimidine can be, for example, Cytosine, Thymine or Uracil, or an analog or precursor of the foregoing pyrimidines. Exemplary pyrimidine precursors include, but are not limited to, dihydroorotate, orotate, uracil monophosphate (UMP), UDP, CMP, or CDP. A pyrimidine analog is any pyrimidine analog that is not an inhibitor of pyrimidine biosynthesis Exemplary pyrimidine analogs include, but are not limited to, Cytosine, Thymine, Uracil, or any of the listed precursors, with one or more substitutions that do not interfere with pyrimidine biosynthesis, cytarabine, nalarabine, sapacitabine, and ARC (4-amino-6-hydrazino-7-β-D-ribofuranosyl-7H-pyrrolo[2,3-d]-pyrimidine-5-carboxamide).

The nucleotide ratio in a cancer to be treated can also be altered by administering to the patient a pyrimidine or a purine base, nucleoside, or nucleotide, in combination with one or more inhibitors of the purine or pyrimidine synthesis pathways, respectively. Inhibitors can be purine or pyrimidine analogs, or inhibitors of critical enzymes in the purine and pyrimidine metabolic pathways.

Pyrimidine bases, nucleosides or nucleotides can be administered with purine analogs that inhibit purine biosynthesis. Exemplary purine analogs that inhibit purine biosynthesis include, but are not limited to, Azathioprine, Mercaptopurine, Clofarabine, Thioguanine, Fludarabine, Pentostatin, Cladribine, 8-amino-adenosine, or Acycloguanosine. Alternatively, pyrimidine bases, nucleosides or nucleotides can be administered with agents that inhibit enzymes critical in purine metabolism. Exemplary inhibitors of the enzyme IMPDH include, but are not limited to, Mercaptopurine, 6-thioguanine, Mycophenolic acid, MMF, and Mizoribine.

Purine bases, nucleosides or nucleotides can be administered with pyrimidine analogs that inhibit pyrimidine biosynthesis. Exemplary pyrimidine analogs that inhibit pyrimidine biosynthesis include, but are not limited to, 5-Fluorouracil (thymidilate synthesis inhibitor), Floxuridine, Cytarabine, 6-azauracil, Gemcitabine, Idoxuridine, 3′-azido-3′deoxythymidine, RX-3117 (flurocyclopentenylcytosine), Pyrazofurin, cytarabine, nalarabine, sapacitabine, and ARC (4-amino-6-hydrazino-7-β-D-ribofuranosyl-7H-pyrrolo[2,3-d]-pyrimidine-5-carboxamide). Alternatively, purine bases, nucleosides, or nucleotides, can be administered with agents that inhibit enzymes critical in pyrimidine metabolism. Exemplary inhibitors of the enzyme DHODH include, but are not limited to Brequinar, Teriflunomide, Leflunomide, Lapachol, Thymidilate synthase, and 5FU, inhibitors of CTP synthase include, but are not limited to, Cyclopentenyl cytosine, inhibitors of UMPS include, but are not limited to 6-azauridine; inhibitors of OMPDC include, but are not limited to, Pyrazofurin; and inhibitors of DHFR include, but are not limited to Pyrimethamine and Methotrexate.

Further, purine or pyrimidine bases, nucleosides or nucleotides can be administered with an inhibitor that affects both the purine and pyrimidine synthesis pathways. Exemplary inhibitors that target GARFT include, but are not limited to the antifolate, lometrexol.

To change the internal baseline ratio of purine:pyrimidine in the cancer, the patient can be treated with a nucleoside, comprising a purine. Examples of such nucleosides are Adenosine comprising Adenine, Deoxyadenosine comprising adenine, Guanosine comprising Guanine, or Deoxyguanosine comprising Guanine. Alternatively, the patient can be treated with a nucleotide comprising a nucleoside comprising a purine. Exemplary nucleotides are Adenylate comprising Adenosine, Deoxyadenylate comprising Deoxyadenosine, Guanylate comprising Guanosine, or Deoxyguanylate comprising Deoxyguanosine. In another example, the patient is treated with a nucleoside comprising a pyrimidine. Examples of such nucleosides are Cytidine comprising Cytosine, Deoxycytidine comprising Cytosine, Thymidine comprising Thymine, Deoxythymidine comprising Thymine, or Uracil comprising Uridine. In yet another example, the patient is treated with a nucleotide comprising a nucleoside comprising a pyrimidine. Exemplary nucleotides are Cytidylate comprising Cytidine, Deoxycytidylate comprising Deoxycytidine, Thymidylate comprising Thymidine, Deoxythymidinylate comprising Deoxythymidine, or Uridylate comprising Uridine.

The cancer to be treated may be a liquid cancer. Liquid cancers are cancers of the cells in the circulatory system. Examples of liquid cancers are, but are not limited to Acute lymphoblastic leukemia (ALL), Chronic lymphocytic leukemia (CLL), Acute myelogenous leukemia (AML), Chronic myelogenous leukemia (CML), Hairy cell leukemia (HCL), T-cell prolymphocytic leukemia (T-PLL), Large granular lymphocytic leukemia, Adult T-cell leukemia, and Clonal eosinophilias. The cancer cell may be in a mammalian species such as, but not limited to a human, monkey, dog, cat, cow, pig, horse, rabbit, rat, or a mouse. Exemplary liquid cancer cell lines include, but are not limited to the human leukemia cancer cell line U937.

The cancer to be treated may be a solid cancer. Solid cancer cells may originate in any of the following organs including, but not limited to bone and muscle; brain; eye, breast; endocrine system including the thyroid; genitourinary tract including kidneys, ovaries, penis, prostate, testicles, urethra, vagina; head and neck including esophagus, nasopharynx, tongue, salivary gland; skin; and the thorax and respiratory tract including lungs, larynx. Exemplary solid cancer cell lines include, but are not limited to the adenocarcinomic human alveolar basal epithelial cell line A549, the osteosarcoma cell line 143B, the human non-small cell lung carcinoma cell line H1299, the human ovarian cancer cell line A2780, the osteosarcoma cell line U205, and the breast cancer cell line MDA-MB-468.

### V. Pharmaceutical Formulations

The purine, a purine analog that is not an inhibitor of purine biosynthesis, a pyrimidine, or a pyrimidine analog that is not an inhibitor of pyrimidine biosynthesis may be administered as a formulation, including a pharmaceutically acceptable carrier may be administered as a formulation, including a pharmaceutically acceptable carrier A patient suffering from or potentially having a cancer or other proliferative disorder can be treated with a purine, pyrimidine, purine analog that is not an inhibitor of purine biosynthesis, and/or pyrimidine analog that is not an inhibitor of pyrimidine biosynthesis which has been formulated for in vivo delivery. Such a formulation comprises a therapeutically effective amount of an amount of a purine, pyrimidine, purine analog, and/or pyrimidine analog which changes the internal baseline purine:pyrimidine ratio in the cancer cells, as well as a pharmaceutically acceptable carrier.

A “therapeutically effective amount” as used herein refers to that amount a purine, pyrimidine, purine precursor, pyrimidine precursor, purine analog, and/or pyrimidine analog which treats, kills, and/or controls the growth and/or metastasis of a tumor or cancer affecting the patient, which inhibits or reduces at least one symptom of the cancer, in addition to changing the internal purine:pyrimidine ration of that cancer.

Pharmaceutical formulation is a well-established art, and is further described, e.g., in Gennaro (ed.), Remington. The Science and Practice of Pharmacy, 20th ed., Lippincott, Williams & Wilkins (2000) (ISBN: 0683306472); Ansel et al., Pharmaceutical Dosage Forms and Drug Delivery Systems, 7th Ed, Lippincott Williams & Wilkins Publishers (1999) (ISBN: 0683305727); and Kibbe (ed,), Handbook of Pharmaceutical. Excipients American Pharmaceutical Association, 3rd Ed. (2000) (ISBN: 091733096X).

The pharmaceutical formulations according to the disclosure further comprise a pharmaceutically acceptable carrier. The term “pharmaceutically acceptable carrier” is to be understood herein as referring to any substance that may, medically, be acceptably administered to a patient, together with purine, pyrimidine, purine precursor, pyrimidine precursor, purine analog, and/or pyrimidine analog according to the disclosure, and which does not undesirably affect the pharmacological activity thereof; a “pharmaceutically acceptable carrier” may thus be, for example, a pharmaceutically acceptable member(s) comprising of diluents, preservatives, solubilizers, emulsifiers, adjuvant, tonicity modifying agents, buffers as well as any other physiologically acceptable vehicle. These formulations are prepared with the pharmaceutically acceptable carrier in accordance with known techniques, for example, those described in Remington, The Science and Practice of Pharmacy (9th Ed. 1995).

The, pharmaceutical formulations can be in a variety of forms. These include, for example, liquid, semi-solid and solid dosage forms, such as liquid solutions (e.g., injectable and infusible solutions), dispersions or suspensions, tablets, pills, powders, liposomes and suppositories. The form can depend on the intended mode of administration and therapeutic application. For example, the pharmaceutical formulation can be in the form of injectable or infusible solutions. The formulation may further include excipient materials, such as sodium chloride, sodium dibasic phosphate heptahydrate, sodium monobasic phosphate, and a stabilizer. It can be provided, for example, in a buffered solution at a suitable concentration and can be stored at 2-8° C.

Such formulations can be administered by a parenteral mode (e.g., intravenous, subcutaneous, intraperitoneal, or intramuscular injection). The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The purine, pyrimidine, purine analog, purine precursor, pyrimidine precursor, and/or pyrimidine analog-containing formulation can be prepared as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The formulation can optionally include other therapeutic agents that treat the cancer or proliferative disorder. Such therapeutic agent include, but are not limited to, aminoglutethimide, amsacrine, anastrozole, asparaginase, Bacillus Calmette-Guerin vaccine (beg), bicalutamide, bleomycin, bortezomib, buserelin, busulfan, campothecin, capecitabine, carboplatin, carfilzomib, carmustine, chlorambucil, chloroquine, cisplatin, cladribine, clodronate, colchicine, cyclophosphamide, cyproterone, cytarabine, dacarbazine, dactinomycin, daunorubicin, demethoxyviridin, dexamethasone, dichloroacetate, dienestrol, diethylstilbestrol, docetaxel, doxorubicin, epirubicin, estradiol, estramustine, etoposide, everolimus, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, letrozole, leucovorin, leuprolide, levamisole, lomustine, lonidamine, mechlorethamine, medroxyprogesterone, megestrol, melphalan, mercaptopurine, mesna, metformin, methotrexate, mitomycin, mitotane, mitoxantrone, nilutamide, nocodazole, octreotide, oxaliplatin, paclitaxel, pamidronate, pentostatin, perifosine, plicamycin, porfimer, procarbazine, raltitrexed, rituximab, sorafenib, streptozocin, sunitinib, suramin, tamoxifen, temozolomide, temsirolimus, teniposide, testosterone, thalidomide, thioguanine, thiotepa, titanocene dichloride, topotecan, trastuzumab, tretinoin, vinblastine, vincristine, vindesine, or vinorelbine.

The formulation can be prepared with ingredients that protect the purine, pyrimidine, purine precursor, pyrimidine precursor, purine analog, and/or pyrimidine analog from rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known (see, e.g., Sustained and Controlled Release Drug Delivery Systems, J. R. Robinson, ed., Marcel Dekker, Inc., New York (1978)).

The pharmaceutical formulation may be prepared for injectable use, topical use, oral use, intramuscular or intravenous injection, inhalation use, transdermal use, transmembrane use, and the like.

These formulations are in unit dosage forms such as tablets, pills, capsules, powders, granules, sterile parenteral solutions or suspensions, metered aerosol or liquid sprays, drops, ampoules, auto-injector devices or suppositories; for oral parenteral, intranasal, sublingual topical or rectal administration, or for administration by inhalation or insufflation. Alternatively, the formulations may be presented in a form suitable for one-weekly or once-monthly administration; for example, an insoluble salt of the active compound, such as decanoate salt, may be adapted to provide a depot preparation for intramuscular injection. An erodible polymer containing the active ingredient may be envisaged.

For preparing solid formulations such as tablets, the purine, pyrimidine, purine precursor, pyrimidine precursor, purine analog, and/or pyrimidine analog is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of purine, pyrimidine, purine analog, and/or pyrimidine analog described herein. Such formulation may be readily subdivided into equally effective unit dosage forms such as tablets, pills and capsules. A therapeutically effective dosage of purine, pyrimidine, purine analog, and/or pyrimidine analog according to the disclosure which treats cancer may vary from patient to patient, and may depend upon factors such as the age of the patient, the patient's genetics, and the diagnosed condition of the patient, and the route of delivery of the dosage form to the patient. A therapeutically effective dose and frequency of administration of a dosage form may be determined in accordance with routine pharmacological procedures known to those skilled in the art. For example, dosage amounts and frequency of administration may vary or change as a function of time and severity of the disorder. A dosage from about 0.1 mg/kg to 10 g/kg, or from about 1 mg/kg to about 10 g/kg may be suitable.

A solid formulation can be subdivided into unit dosage forms of the type described above containing from 0.1 mg to about 1 g or about 1 mg to about 500 mg of the agent (purine, purine precursor, purine analog, pyrimidine, pyrimidine precursor, and/or pyrimidine analog). Some useful, nonlimiting unit dosage forms contain from 1 to 100 mg, for example 1 mg, 2 mg, 5 mg, 10 mg, 25 mg, 50 mg, or 100 mg, of the agent. The tablets or pills of the formulation can be coated or otherwise compounded to provide a dosage form affording the advantage of prolonged action. The liquid forms in which the agent may be incorporated for administration orally or by injection include aqueous solutions, suitably flavored syrups, aqueous or oil suspensions, and flavored emulsions with edible oils as well as elixirs and similar pharmaceutical vehicles. In the treatment of cancer, a suitable dosage level of agent is about 0.001 mg/kg to about 250 mg/kg per day. The formulations may be administered as a bolus, as a regimen of 1 to about 4 times per day, or as a continuous infusion.

Injectable dosage forms may be sterilized in a pharmaceutically acceptable fashion, for example by steam sterilization of an aqueous solution sealed in a vial under an inert gas atmosphere at 120° C. for about 15 minutes to 20 minutes, or by sterile filtration of a solution through a 0.2 μM or smaller pore-size filter, optionally followed by a lyophilization step, or by irradiation of a formulation containing a derivative of the present disclosure by means of emissions from a radionuclide source.

Reference will now be made to specific examples illustrating the disclosure. It is to be understood that the examples are provided to illustrate exemplary embodiments and that no limitation to the scope of the disclosure is intended thereby.

### EXAMPLES

### Example 1

### Differentiation of Leukemia Cells with Nucleotides

### Proliferation Rates

All adherent and human leukemia cell lines were cultured in DMEM or RPMI supplemented with 10% heat inactivated fetal bovine serum at 37° C. with 5% CO₂. The Lys-GFP-ER-HoxA9 63.3 cells were cultured in RPMI supplemented with 10% fetal bovine serum, stem cell factor, and beta-estradiol as described previously (Sykes et al., (2016), Cell. 167(1): 171-186.e15). All cell lines regularly tested negative for mycoplasma.

To assess proliferation, the number of cells seeded for each cell line allowed for exponential growth over the course of the assay. To assess effects of nucleotide treatment, cells were washed three times with PBS and 4 mL of treatment media was added one day after plating cells. The following formula was used to calculate proliferation rate:

Doublings per day=[Log₂(Final day 4 cell count/Initial day 0 cell count)]/4 days

### Assessment of Differentiation

The Lys-GFP-ER-HoxA9 cells or human leukemia cell lines were grown in round-bottom tissue-culture treated plates. Following a 4-day incubation with nucleotides, cells were incubated with antibodies, washed, incubated with FACS buffer containing DAPI, and analyzed by flow cytometry. Viability was assessed by forward and side scatter as well as by DAPI exclusion. For the Lys-GFP-ER-HoxA9 63.3 cells, differentiation was assessed by GFP and staining with an anti-CD11b-APC antibody (final concentration of 1:400; Clone M1/70, Biolegend). For the human leukemia cell lines, differentiation was assessed with the anti-CD11b-APC antibody and an anti-CD16-Alexa Fluor 488 antibody (final concentration of 1:400; Clone 3G8, BioLegend).

### LCMS Analysis

Where indicated, cells were incubated in media with 4 mM ¹⁵N-amide-glutamine and/or 200 μM ¹³C-guanine or ¹³C-adenine for 24 hours prior to LCMS Analysis of metabolites. For assessment of metabolites, cells were washed three times with PBS prior to extracting polar metabolites from cells: plates were placed on ice, cells were washed with ice-cold blood bank saline, and 500 μl of ice-cold 80% methanol in water with 250 nM ¹³C/¹⁵N labeled amino acid standards (MSK-A2-1.2: Cambridge Isotope Laboratories, Inc.) was added to each well. Cells were scraped, each sample was vortexed for 10 minutes at 4° C., and then centrifuged at maximum speed for 10 minutes at 4° C. Samples were dried under nitrogen gas and resuspended in 25 μl of a 50/50 acetonitrile/water mixture. Metabolites were measured using a Dionex UltiMate 3000 ultra-high performance liquid chromatography system connected to a Q Exactive benchtop Orbitrap mass spectrometer, equipped with an Ion Max source and a HESI II probe (Thermo Fisher Scientific). Samples were separated by chromatography by injecting 2-10 μl of sample on a SeQuant ZIC-pHILIC Polymeric column (2.1×150 mm 5 μM, EMD Millipore). Flow rate was set to 150 μl/min, temperatures were set to 25° C. for column compartment and 4° C. for autosampler sample tray. Mobile Phase A consisted of 20 mM ammonium carbonate, 0.1% ammonium hydroxide. Mobile Phase B was 100% acetonitrile. The mobile phase gradient (% B) was set in the following protocol: 0-20 min.: linear gradient from 80% to 20% B; 20-20.5 min.: linear gradient from 20% to 80% B; 20.5-28 min.: hold at 80% B. Mobile phase was introduced into the ionization source set to the following parameters: sheath gas=40, auxiliary gas=15, sweep gas=1, spray voltage=−3.1 kV, capillary temperature=275° C., S-lens RF level=40, probe temperature=350° C.

Metabolites were monitored in full-scan, polarity-switching, mode. An additional narrow range full-scan (220-700 m/z) in negative mode only was included to enhance nucleotide detection. The resolution was set at 70,000, the AGC target at 1,000,000, and the maximum injection time at 20 msec. Relative quantitation of metabolites was performed with XCalibur QuanBrowser 2.2 (Thermo Fisher Scientific) using a 5 ppm mass tolerance and referencing an in-house retention time library of chemical standards. Metabolite measurements were normalized to the internal ¹³C/¹⁵N labeled amino acid standard and to cell number allowing us to assess levels of each nucleotide as shown in FIG. 4.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A method of inhibiting proliferation in a cancer cell, the cancer cell having an endogenous baseline ratio of purine:pyrimidine, the method comprising:
contacting the cell with an agent in an amount sufficient to change the endogenous baseline ratio of purine:pyrimidine in the cell, resulting in a nucleotide imbalance,
the agent comprising a purine, a purine precursor, a purine analog that is not a purine biosynthesis inhibitor, a pyrimidine, a pyrimidine precursor, and/or a pyrimidine analog that is not a pyrimidine biosynthesis inhibitor,
the nucleotide imbalance inhibiting the cancer cell from proliferating.

2. The method of claim 1, wherein the agent is a purine nucleotide.

3. The method of claim 2, wherein the purine nucleotide is Adenine or Guanine.

4. The method of claim 1, wherein the agent is Adenine.

5. The method of claim 1, wherein the agent is a purine precursor.

6. The method of claim 5, wherein the purine precursor is AIR, CAIR, SACAIR, AICAR, FAICAR inosine mono phosphate (IMP), adenylosuccinate, xanthine, or hypoxanthine.

7. The method of claim 1, wherein the agent is a purine analog that does not inhibit purine biosynthesis.

8. The method of claim 7, wherein the purine analog is 8-amino-adenosine.

9. The method of claim 1, wherein the cancer cell is contacted with at least one purine and at least one purine precursor.

10. The method of claim 9, wherein the cancer cell is further contacted with at least on purine analog that is not an inhibitor of purine biosynthesis.

11. The method of claim 1, further comprising contacting the cancer cell with a pyrimidine biosynthesis inhibitor.

12. The method of claim 11, wherein the pyrimidine biosynthesis inhibitor is mercaptopurine, 6-mercaptopurine, mycophenolic acid, mycophenolate mofetil, 6-thioguanine, lometrexol, pyrimethamine, or cladribine.

13. The method of claim 1, wherein the agent is a pyrimidine nucleotide.

14. The method of claim 13, wherein the pyrimidine nucleotide is Cytosine, Thymidine, or Uracil.

15. The method of claim 1, wherein the agent is a pyrimidine precursor.

16. The method of claim 15, wherein the pyrimidine precursor is dihydroorotate, orotate, uracil monophosphate (UMP), UDP, CMP, or CDP.

17. The method of claim 1, wherein the agent is a pyrimidine analog that does not inhibit pyrimidine biosynthesis.

18. The method of claim 17, wherein the pyrimidine analog is cytarabine, nalarabine, sapacitabine, ARC (4-amino-6-hydrazino-7-β-D-ribofuranosyl-7H-pyrrolo[2,3-d]-pyrimidine-5-carboxamide).

19. The method of claim 1, wherein the cancer cell is contacted with at least one pyrimidine and at least one pyrimidine precursor.

20. The method of claim 19, wherein the cancer cell is further contacted with at least on pyrimidine biosynthesis inhibitor that is not a pyrimidine biosynthesis inhibitor.

21. The method of claim 1, further comprising contacting the cancer cell with a purine biosynthesis inhibitor.

22. The method of claim 21, wherein the purine biosynthesis inhibitor is Azathioprine, Mercaptopurine, Clofarabine, Thioguanine, Fludarabine, Pentostatin, Cladribine or Acycloguanosine.

23. The method of claim 1, further comprising contacting the cells with a pyrimidine biosynthesis inhibitor.

24. The method of claim 23, wherein the pyrimidine biosynthesis inhibitor is brequinar, leflunomide, teriflunomide, pyrazofurin, cyclopentenyl cytosine, fluorocyclopentenylcytosine, 5-fluorouracil, ralitrexed, pemetrexed, or 6-azauridine.

25. The method of claim 1, wherein the cancer cell is a liquid cancer cell or a solid cancer cell.

26. A method of inducing differentiation in a cancer cell, the cancer cell having an endogenous baseline ratio of purine:pyrimidine, the method comprising:
contacting the cell with an agent in an amount sufficient to change the endogenous baseline ratio of purine:pyrimidine in the cell, resulting in a nucleotide imbalance,
the agent comprising a purine, a purine precursor, a purine analog that is not a purine biosynthesis inhibitor, a pyrimidine, a pyrimidine precursor, and/or a pyrimidine analog that is not a pyrimidine biosynthesis inhibitor,
the nucleotide imbalance inducing differentiation in the cancer cell.

27. The method of claim 26, wherein the agent is a purine nucleotide.

28. The method of claim 27, wherein the purine nucleotide is Adenine or Guanine.

29. The method of claim 28, wherein the agent is Adenine.

30. The method of claim 26, wherein the agent is a purine precursor.

31. The method of claim 30, wherein the purine precursor is AIR, CAIR, SACAIR, AICAR, FAICAR inosine mono phosphate (IMP), adenylosuccinate, xanthine, or hypoxanthine.

32. The method of claim 26, wherein the agent is a purine analog that is not a purine biosynthesis inhibitor.

33. The method of claim 32, wherein the purine analog is 8-amino-adenosine.

34. The method of claim 26, wherein the cancer cell is contacted with at least one purine and at least one purine precursor.

35. The method of claim 34, wherein the cancer cell is further contacted with at least on purine analog that is not an inhibitor of purine biosynthesis.

36. The method of claim 26, further comprising contacting the cancer cell with a pyrimidine biosynthesis inhibitor.

37. The method of claim 36, wherein the pyrimidine biosynthesis inhibitor is mercaptopurine, 6-mercaptopurine, mycophenolic acid, mycophenolate mofetil, 6-thioguanine, lometrexol, pyrimethamine, or cladribine.

38. The method of claim 26, wherein the agent is a pyrimidine nucleotide.

39. The method of claim 38, wherein the pyrimidine nucleotide is Cytosine, Thymidine, or Uracil.

40. The method of claim 27 wherein the agent is a pyrimidine precursor.

41. The method of claim 40, wherein the pyrimidine precursor is dihydroorotate, orotate, uracil monophosphate (UMP), UDP, CMP, or CDP.

42. The method of claim 26, wherein the agent is a pyrimidine analog that is not an inhibitor of pyrimidine biosynthesis.

43. The method of claim 42, wherein the pyrimidine analog is cytarabine, nalarabine, sapacitabine, ARC (4-amino-6-hydrazino-7-β-D-ribofuranosyl-7H-pyrrolo[2,3-d]-pyrimidine-5-carboxamide).

44. The method of claim 26, wherein the cancer cell is contacted with at least one pyrimidine and at least one pyrimidine precursor.

45. The method of claim 44, wherein the cancer cell is further contacted with at least on pyrimidine analog that is not an inhibitor of pyrimidine biosynthesis.

46. The method of claim 26, further comprising contacting the cell with a purine biosynthesis inhibitor.

47. The method of claim 46, wherein the purine biosynthesis inhibitor is Azathioprine, Mercaptopurine, Clofarabine, Thioguanine, Fludarabine, Pentostatin, Cladribine, or Acycloguanosine.

48. The method of claim 26, further comprising contacting the cancer cell with a pyrimidine biosynthesis inhibitor.

49. The method of claim 48, wherein the pyrimidine biosynthesis inhibitor is brequinar, leflunomide, teriflunomide, pyrazofurin, cyclopentenyl cytosine, fluorocyclopentenylcytosine, 5-fluorouracil, ralitrexed, pemetrexed, or 6-azauridine.

50. The method of claim 26, wherein the cancer cell is a liquid cancer cell or a solid cancer cell.

51. A method of inducing replication stress in a cancer cell, the cancer cell having an endogenous baseline ratio of purine:pyrimidine, the method comprising:
contacting the cell with an agent in an amount sufficient to change the endogenous baseline ratio of purine:pyrimidine in the cell, resulting in a nucleotide imbalance,
the agent comprising a purine, a purine precursor, a purine analog that is not a purine biosynthesis inhibitor, a pyrimidine, a pyrimidine precursor, and/or a pyrimidine analog that is not a pyrimidine biosynthesis inhibitor,
the nucleotide imbalance inducing replication stress in the cancer cell.

52. The method of claim 51, wherein the agent is a purine nucleotide.

53. The method of claim 52, wherein the purine nucleotide is Adenine or Guanine.

54. The method of claim 53, wherein the agent is Adenine.

55. The method of claim 51, wherein the agent is a purine precursor.

56. The method of claim 55, wherein the purine precursor is AIR, CAIR, SACAIR, AICAR, FAICAR inosine mono phosphate (IMP), adenylosuccinate, xanthine, and hypoxanthine.

57. The method of claim 51, wherein the agent is a purine analog that is not a purine biosynthesis inhibitor.

58. The method of claim 57, wherein the purine analog is 8-amino-adenosine.

59. The method of claim 51, wherein the cancer cell is contacted with at least one purine, and at least one purine precursor.

60. The method of claim 59, wherein the cancer cell is further contacted with at least on purine analog that is not an inhibitor of purine biosynthesis.

61. The method of claim 51, further comprising contacting the cancer cell with a pyrimidine biosynthesis inhibitor.

62. The method of claim 61, wherein the pyrimidine biosynthesis inhibitor is mercaptopurine, 6-mercaptopurine, mycophenolic acid, mycophenolate mofetil, 6-thioguanine, lometrexol, pyrimethamine, or cladribine.

63. The method of claim 51, wherein the agent is a pyrimidine nucleotide.

64. The method of claim 63, wherein the pyrimidine nucleotide is Cytosine, Thymidine, or Uracil.

65. The method of claim 51, wherein the agent is a pyrimidine precursor.

66. The method of claim 65, wherein the pyrimidine precursor is dihydroorotate, orotate, uracil monophosphate (UMP), UDP, CMP, or CDP.

67. The method of claim 51, wherein the agent is a pyrimidine analog that is not a pyrimidine biosynthesis inhibitor.

68. The method of claim 67, wherein the pyrimidine analog is cytarabine, nalarabine, sapacitabine, ARC (4-amino-6-hydrazino-7-β-D-ribofuranosyl-7H-pyrrolo[2,3-d]-pyrimidine-5-carboxamide).

69. The method of claim 51, further comprising contacting the cancer cell with a purine biosynthesis inhibitor.

70. The method of claim 69, wherein the purine biosynthesis inhibitor is brequinar, leflunomide, teriflunomide, pyrazofurin, cyclopentenyl cytosine, fluorocyclopentenylcytosine, 5-fluorouracil, ralitrexed, pemetrexed, or 6-azauridine

71. The method of claim 51, wherein the cancer cell is contacted with at least one pyrimidine and at least one pyrimidine precursor.

72. The method of claim 71, wherein the cancer cell is further contacted with at least on pyrimidine analog that is not a purine biosynthesis inhibitor.

73. The method of claim 72, wherein the pyrimidine analog is cytarabine, nalarabine, sapacitabine, ARC (4-amino-6-hydrazino-7-β-D-ribofuranosyl-7H-pyrrolo[2,3-d]-pyrimidine-5-carboxamide).

74. The method of claim 51, further comprising contacting the cell with a purine synthesis inhibitor.

75. The method of claim 74, wherein the purine biosynthesis inhibitor is Azathioprine, Mercaptopurine, Clofarabine, Thioguanine, Fludarabine, Pentostatin, Cladribine, or Acycloguanosine

76. The method of claim 51, wherein the cancer cell is a liquid cancer cell or a solid cancer cell.

77. A method of treating a subject afflicted with a cancer, comprising
administering to the subject a therapeutically effective amount of an agent that changes the endogenous baseline purine:pyrimidine ratio in a cell of the cancer, thereby causing a nucleotide imbalance in the cancer,
the agent comprising a purine, a purine precursor, a purine analog that is not a purine biosynthesis inhibitor, a pyrimidine, a pyrimidine precursor, and/or a pyrimidine analog that is not a pyrimidine biosynthesis inhibitor,
the nucleotide imbalance resulting in a reduction in, and/or inhibition of proliferation of the cancer.

78. The method of claim 77, wherein the agent is a purine nucleotide.

79. The method of claim 78, wherein the purine nucleotide is Adenine or Guanine.

80. The method of claim 79, wherein the agent is Adenine.

81. The method of claim 77, wherein the agent is a purine precursor.

82. The method of claim 81, wherein the purine precursor is AIR, CAIR, SACAIR, AICAR, FAICAR inosine mono phosphate (IMP), adenylosuccinate, xanthine, or hypoxanthine

83. The method of claim 77, wherein the agent is a purine analog that is not a purine biosynthesis inhibitor.

84. The method of claim 83, wherein the purine analog is 8-amino-adenosine.

85. The method of claim 77, wherein the cancer cell is contacted with at least one purine, and at least one purine precursor.

86. The method of claim 85, wherein the cancer cell is further contacted with at least on purine analog that is not an inhibitor of purine biosynthesis.

87. The method of claim 77, further comprising contacting the cancer cell with a pyrimidine biosynthesis inhibitor.

88. The method of claim 87, wherein the pyrimidine biosynthesis inhibitor is mercaptopurine, 6-mercaptopurine, mycophenolic acid, mycophenolate mofetil, 6-thioguanine, lometrexol, pyrimethamine, or cladribine

89. The method of claim 77, wherein the agent is a pyrimidine nucleotide.

90. The method of claim 89, wherein the pyrimidine nucleotide is Cytosine, Thymidine, or Uracil.

91. The method of claim 77, wherein the agent is a pyrimidine precursor.

92. The method of claim 91, wherein the pyrimidine precursor is dihydroorotate, orotate, uracil monophosphate (UMP), UDP, CMP, or CDP.

93. The method of claim 77, wherein the agent is a pyrimidine analog that does not affect pyrimidine biosynthesis.

94. The method of claim 93, wherein the pyrimidine analog is cytarabine, nalarabine, sapacitabine, ARC (4-amino-6-hydrazino-7-β-D-ribofuranosyl-7H-pyrrolo[2,3-d]-pyrimidine-5-carboxamide).

95. The method of claim 77, wherein the cancer cell is contacted with at least one pyrimidine and at least one pyrimidine precursor.

96. The method of claim 95, further comparing contacting the cell with at least one pyrimidine analog that is not a pyrimidine biosynthesis inhibitor.

97. The method of claim 77, wherein the cancer cell is further contacted with at least one purine biosynthesis inhibitor.

98. The method of claim 97, wherein the purine biosynthesis inhibitor is Azathioprine, Mercaptopurine, Clofarabine, Thioguanine, Fludarabine, Pentostatin, Cladribine or Acycloguanosine.

99. The method of claim 77, wherein the cancer is a liquid cancer or a solid cancer.

100. The method of claim 77, wherein the agent is in a formulation.
